(19) United States
(12) Patent Application Publication
(10) Pub. No.: US 2026/0253742 A1
(43) Pub. Date: Aug. 27, 2026

(54) SYSTEM

(71) Applicant: SoftBank Group Corp., Tokyo (JP)

(72) Inventor: Ryoichi ASANO, Tokyo (JP)

(73) Assignee: SoftBank Group Corp., Tokyo (JP)

(21) Appl. No.: 19/543,875

(22) Filed: Feb. 19, 2026

(30) Foreign Application Priority Data
Feb. 21, 2025 (JP) 2025-027006

(51) Int. Cl. G16H 50/30; G06V 10/82; G06V 40/16; G10L 15/08; G10L 15/16; G10L 25/63; G16H 10/60; G16H 40/67; G16H 50/20

(52) U.S. Cl. CPC G16H 50/30; G06V 10/82; G06V 40/174; G10L 15/16; G10L 25/63; G16H 10/60; G16H 40/67; G16H 50/20; G10L 2015/088

(57) ABSTRACT

The system according to the embodiment comprises a receiving unit, an estimation unit, a display unit, an analysis unit, and a proposal unit. The receiving unit is configured to receive physical condition information of a user. The estimation unit is configured to analyze the physical condition information received by the receiving unit and estimate a disease name. The display unit is configured to display a hospital to be visited based on the disease name estimated by the estimation unit. The analysis unit is configured to analyze examination results after the visit. The proposal unit is configured to propose a hospital to be referred to based on the results analyzed by the analysis unit.

### CROSS-REFERENCE TO RELATED APPLICATION

The present application claims priority to and incorporates by reference the entire contents of Japanese Patent Application No. 2025-027006 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that it is not sufficiently possible to visit an appropriate hospital based on the user's physical condition information or to refer an appropriate hospital based on examination results after the visit.

### SUMMARY OF THE INVENTION

The system according to the embodiment comprises a receiving unit, an estimation unit, a display unit, an analysis unit, and a proposal unit. The receiving unit is configured to receive physical condition information of a user. The estimation unit is configured to analyze the physical condition information received by the receiving unit and estimate a disease name. The display unit is configured to display a hospital to be visited based on the disease name estimated by the estimation unit. The analysis unit is configured to analyze examination results after the visit. The proposal unit is configured to propose a hospital to be referred to based on the results analyzed by the analysis unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

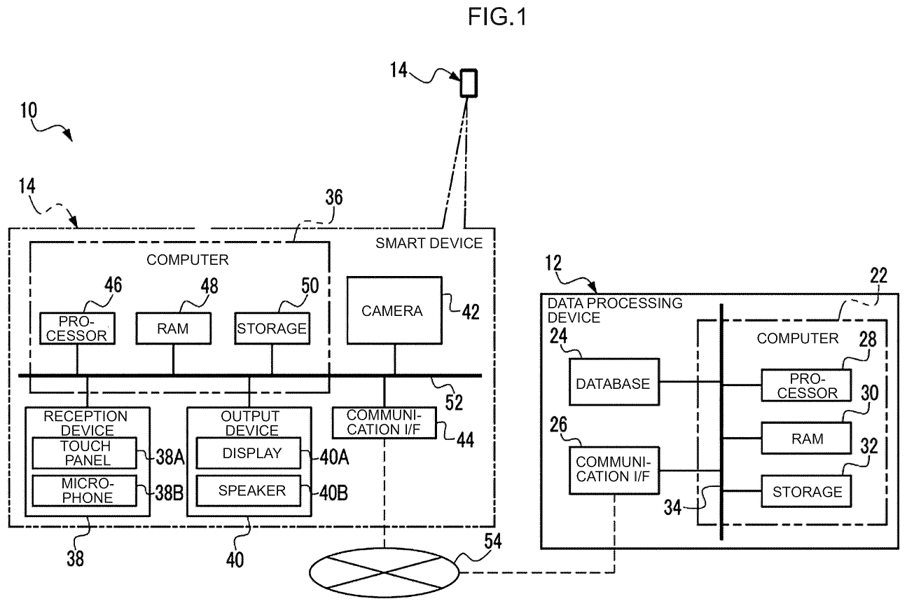

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

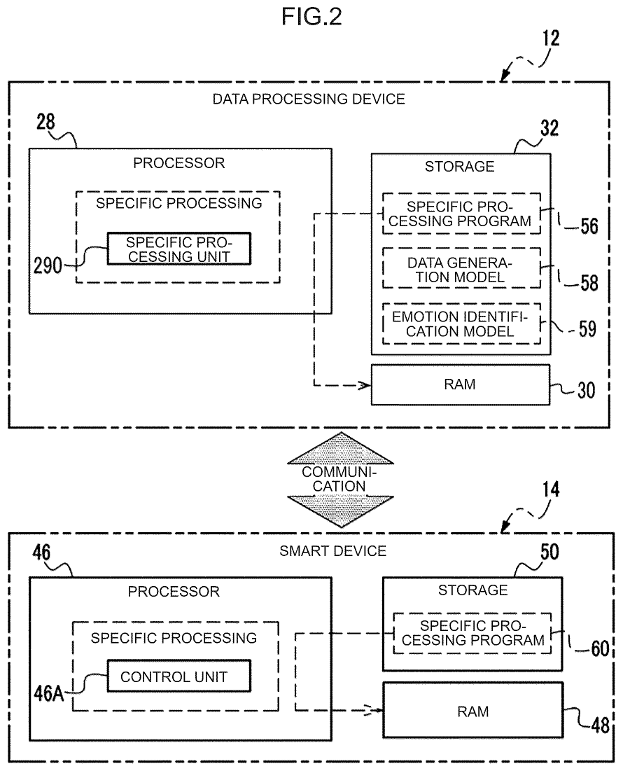

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention is a system that analyzes a user's physical condition information and displays an estimated disease name and a hospital to be visited. This system begins when the user inputs physical conditions such as body temperature, nausea, headache, and the location and degree of pain. Next, the system analyzes this information and displays a list of estimated disease names and hospitals to be visited. Furthermore, after the user visits a hospital, the system analyzes the results of blood tests, MRI, CRI, X-ray, and ultrasound examinations performed there and proposes a hospital to be referred to. This system can reduce the problem of patients being sent from hospital to hospital. For example, when the user inputs physical conditions such as body temperature, nausea, headache, and the location and degree of pain, the system estimates a disease name based on this information and displays a list of hospitals to be visited. After the user visits a hospital, the system analyzes the results of blood tests, MRI, CRI, X-ray, and ultrasound examinations and proposes the optimal hospital. As a result, the user can select an appropriate hospital and receive medical care efficiently. Thus, the system can efficiently analyze the user's physical condition information and propose appropriate disease names and hospitals. Specifically, the system stores the physical condition information input by the user (e.g., body temperature 36.8° C., presence or absence of nausea, presence or absence of headache, pain location: lower right abdomen, pain degree: 7 out of 10, etc.) in a database as numerical vectors or categorical data in the receiving unit. The system normalizes and standardizes these input data in a preprocessing unit, performs missing value imputation and outlier detection, and then inputs them to the estimation unit. The estimation unit, for example, uses a multilayer perceptron, convolutional neural network, or Transformer-based large language model to receive the physical condition information vector as an input tensor (e.g., 1×N dimensions, where N is the number of items) and generates as output a label list of candidate disease names (e.g., gastroenteritis, migraine, influenza, etc.) and probability scores for each disease name (e.g., gastroenteritis 0.65, migraine 0.20, influenza 0.10, etc.). Furthermore, the estimation unit sends a request to the display unit for hospital selection based on the output candidate disease names and probability scores. The display unit refers to structured data such as specialty (e.g., gastroenterology, neurology, etc.), distance to hospital, past treatment records, and patient evaluation scores for each disease name and generates an optimal hospital list. After the user visits a hospital, the results of blood tests (e.g., numerical vectors such as white blood cell count, CRP value, hemoglobin value, etc.), MRI and CRI (image data as 3D tensors, e.g., 256×256×128), X-ray and ultrasound (2D image tensors, e.g., 512×512), and other examination results are individually preprocessed (noise removal, feature extraction, image segmentation, etc.) by the analysis unit and then input to an AI model (e.g., CNNs such as ResNet or U-Net, or multimodal fusion models). The analysis unit estimates abnormal findings (e.g., anemia, infection, cerebral infarction, fracture, liver tumor, etc.) from the examination results and generates structured data such as abnormal site labels, severity scores, and recommended treatment policies as output. The proposal unit refers to a specialist database (e.g., department, treatment records, patient evaluation, accessibility, etc.) based on these analysis results, identifies the optimal hospital for the user's symptoms and examination results, and adjusts the proposal order and level of detail for display. This series of processes, unlike conventional human interviews and referral work, is executed by AI combining feature extraction in high-dimensional vector space, probabilistic inference, and rule-based branching, resulting in significant improvements in processing speed, estimation accuracy, reduction of misdiagnosis risk, efficiency of data management, and optimization of communication load, thereby improving computer technology itself. Specific application fields include triage support in emergency medical settings, telemedicine platforms, chronic disease management systems, and corporate health management support services.

The system according to the embodiment comprises a receiving unit, an estimation unit, a display unit, an analysis unit, and a proposal unit. The receiving unit receives physical condition information of the user. The user's physical condition information may include, for example, body temperature, blood pressure, heart rate, and details of symptoms, but is not limited to such examples. The receiving unit records information such as body temperature, nausea, headache, etc., input by the user. The receiving unit can also record the user's physical condition information in detail. For example, the receiving unit stores the physical condition information input by the user in a database for later analysis. The estimation unit analyzes the physical condition information received by the receiving unit and estimates a disease name. The estimation unit, for example, uses AI to analyze the physical condition information and estimate a disease name. The estimation unit can estimate a disease name by analyzing physical conditions such as body temperature, nausea, headache, and the location and degree of pain. For example, if the body temperature is high, the estimation unit estimates a disease accompanied by fever; if nausea is present, it estimates a gastrointestinal disease. The display unit displays a hospital to be visited based on the disease name estimated by the estimation unit. For example, the display unit displays a list of hospitals to be visited by the user based on the estimated disease name. The display unit can select a hospital by considering the specialty, distance, and evaluation of the hospital based on the estimated disease name. The analysis unit analyzes examination results after the visit. For example, the analysis unit analyzes results of blood tests, MRI, CRI, X-ray, and ultrasound examinations. The analysis unit can use AI to analyze examination results and estimate a disease name or treatment policy. For example, the analysis unit estimates anemia or infection from blood test results and estimates abnormalities in the brain or spinal cord from MRI results. The proposal unit proposes a hospital to be referred to based on the results analyzed by the analysis unit. For example, the proposal unit identifies a hospital based on specialist data. The proposal unit can propose a hospital to be visited by the user based on the results analyzed by the analysis unit. For example, the proposal unit proposes an internal medicine specialist based on blood test results and a neurology specialist based on MRI results. Thus, the system according to the embodiment can efficiently analyze the user's physical condition information and propose appropriate disease names and hospitals. Specifically, the system stores the physical condition information received by the receiving unit from the user (e.g., body temperature 36.8° C., blood pressure 120/80 mmHg, heart rate 78 bpm, presence or absence of nausea, presence or absence of headache, pain location: lower right abdomen, pain degree: 7 out of 10, etc.) in a database as numerical vectors or categorical data. The system normalizes and standardizes these input data in a preprocessing unit, performs missing value imputation and outlier detection (e.g., outlier removal, median imputation, Z-score judgment, etc.), and then inputs them to the estimation unit. The estimation unit, for example, uses a multilayer perceptron, convolutional neural network, or Transformer-based large language model to receive the physical condition information vector as an input tensor (e.g., 1×N dimensions, where N is the number of items) and generates as output a label list of candidate disease names (e.g., gastroenteritis, migraine, influenza, etc.) and probability scores for each disease name (e.g., gastroenteritis 0.65, migraine 0.20, influenza 0.10, etc.). Inside the AI model, techniques such as ReLU activation function, batch normalization, and dropout are combined to perform inference using trained weights. Furthermore, the estimation unit sends a request to the display unit for hospital selection based on the output candidate disease names and probability scores. The display unit refers to structured data such as specialty (e.g., gastroenterology, neurology, etc.), distance to hospital, past treatment records, and patient evaluation scores for each disease name and generates an optimal hospital list. After the user visits a hospital, the results of blood tests (e.g., numerical vectors such as white blood cell count, CRP value, hemoglobin value, etc.), MRI and CRI (image data as 3D tensors, e.g., 256×256×128), X-ray and ultrasound (2D image tensors, e.g., 512×512), and other examination results are individually preprocessed (noise removal, feature extraction, image segmentation, etc.) by the analysis unit and then input to an AI model (e.g., CNNs such as ResNet or U-Net, or multimodal fusion models). The analysis unit estimates abnormal findings (e.g., anemia, infection, cerebral infarction, fracture, liver tumor, etc.) from the examination results and generates structured data such as abnormal site labels, severity scores, and recommended treatment policies as output. The proposal unit refers to a specialist database (e.g., department, treatment records, patient evaluation, accessibility, etc.) based on these analysis results, identifies the optimal hospital for the user's symptoms and examination results, and adjusts the proposal order and level of detail for display. This series of processes, unlike conventional human interviews and referral work, is executed by AI combining feature extraction in high-dimensional vector space, probabilistic inference, and rule-based branching, resulting in significant improvements in processing speed, estimation accuracy, reduction of misdiagnosis risk, efficiency of data management, and optimization of communication load, thereby improving computer technology itself. Specific application fields include triage support in emergency medical settings, telemedicine platforms, chronic disease management systems, and corporate health management support services.

The receiving unit can record the user's physical condition information. For example, the receiving unit stores the physical condition information input by the user in a database. For example, the receiving unit stores information such as body temperature, blood pressure, heart rate, and details of symptoms input by the user in a database. The receiving unit can also record the user's physical condition information periodically. For example, the receiving unit records the physical condition information input by the user daily and tracks long-term changes in physical condition. By recording the user's physical condition information in detail, more accurate analysis becomes possible. Some or all of the above-described processing in the receiving unit may be performed using AI or without using AI. For example, the receiving unit can input the physical condition information entered by the user into AI and have the AI perform the recording of the physical condition information. Specifically, the receiving unit stores the physical condition information input by the user (e.g., body temperature 36.8° C., blood pressure 120/80 mmHg, heart rate 78 bpm, presence or absence of nausea, presence or absence of headache, pain location: lower right abdomen, pain degree: 7 out of 10, etc.) in a database as numerical vectors (e.g., 1×N dimensions, where N is the number of items) or categorical data (e.g., presence/absence, location labels, etc.). The receiving unit sends these input data to the preprocessing unit, which performs normalization (e.g., Z-score normalization), standardization, missing value imputation (e.g., median imputation), and outlier detection (e.g., IQR method or Z-score judgment). When using AI, the receiving unit inputs the physical condition information entered by the user into an AI model such as a large language model or multilayer perceptron, and has the AI perform automatic classification of input values, automatic detection of outliers, automatic correction of input errors, and automatic completion of input items. For example, as an input example to the AI model, vector data such as body temperature 36.8° C., blood pressure 120/80 mmHg, heart rate 78 bpm, nausea present, headache absent, pain location: lower right abdomen, pain degree: 7, etc. can be provided. The AI model outputs normalized vectors of data to be recorded, outlier flags (e.g., outlier flag 1 if body temperature is 39° C.), and input completion candidates (e.g., “lower right abdomen” as a candidate if pain location is not entered). These outputs are used to improve the quality of recorded data and provide high-precision data to subsequent estimation and analysis units. When the receiving unit records the user's physical condition information periodically, it manages the data as a time-series database (e.g., an array of physical condition vectors by date) and utilizes it for trend analysis of long-term changes in physical condition and anomaly detection (e.g., alert if high fever continues for several days). These processes, unlike conventional manual input and recording by humans, realize unconventional high-dimensional data processing such as automatic classification, anomaly detection, data completion, and time-series analysis by AI, resulting in improved recording accuracy, efficiency of data management, supply of high-quality data to the analysis and estimation units, reduction of erroneous input risk, and automation of long-term health management, thereby improving computer technology itself. Specific application fields include remote health management systems, corporate health management support, home monitoring for chronic disease patients, and wearable device-linked health record services.

The analysis unit can analyze results of blood tests, MRI, CRI, X-ray, and ultrasound examinations. For example, the analysis unit analyzes blood test results. For example, the analysis unit estimates anemia or infection from blood test results. The analysis unit can also analyze MRI results. For example, the analysis unit estimates abnormalities in the brain or spinal cord from MRI results. The analysis unit can also analyze CRI results. For example, the analysis unit estimates abnormalities in the heart or blood vessels from CRI results. The analysis unit can also analyze X-ray results. For example, the analysis unit estimates fractures or lung abnormalities from X-ray results. The analysis unit can also analyze ultrasound examination results. For example, the analysis unit estimates abnormalities in internal organs from ultrasound examination results. By analyzing various examination results, more accurate hospital proposals become possible. Some or all of the above-described processing in the analysis unit may be performed using AI or without using AI. For example, the analysis unit can input blood test results into AI and have the AI perform the analysis. Specifically, the analysis unit individually receives blood test results (e.g., numerical vectors such as white blood cell count, CRP value, hemoglobin value, etc., e.g., 1×M dimensions, where M is the number of test items), MRI and CRI image data (e.g., MRI as 3D tensor 256×256×128, CRI as 2D or 3D image tensor), and X-ray and ultrasound image data (e.g., 2D image tensor 512×512). The analysis unit performs preprocessing of these examination data in the preprocessing unit, such as noise removal (e.g., median filter, Gaussian filter), image normalization, feature extraction (e.g., edge detection, region segmentation), and image segmentation (e.g., organ region extraction by CNN such as U-Net). When using AI, the analysis unit inputs blood test vectors into models such as multilayer perceptron or gradient boosting decision trees and generates structured data as output, such as abnormal value labels (e.g., anemia 1, infection 0), severity scores (e.g., 0.8), and recommended treatment policies (e.g., iron supplementation). MRI and CRI images are input into convolutional neural networks (e.g., ResNet, U-Net) or multimodal fusion models, and output abnormal site labels (e.g., cerebral infarction, spinal cord tumor), severity scores (e.g., 0.9), and recommended treatment policies (e.g., surgical treatment). X-ray and ultrasound images are similarly analyzed by CNNs, etc., to estimate abnormal findings such as fractures, pneumonia, and liver tumors. Input examples to the AI model include blood test vectors (white blood cell count 8000, CRP 1.2, hemoglobin 13.5), MRI image tensor (256×256×128), X-ray image tensor (512×512), etc. Output examples from the AI model include abnormal finding labels (anemia 1, infection 0), abnormal site (cerebral infarction), severity score 0.85, recommended treatment policy (internal medicine treatment), etc. These outputs are used for hospital selection and treatment policy proposals in the subsequent proposal unit. The analysis unit can also integratively analyze multiple examination results and perform correlation analysis of abnormal findings and estimation of complex diseases. These processes, unlike conventional visual/manual interpretation of examination results by humans, realize unconventional techniques such as feature extraction in high-dimensional vector space, probabilistic inference, rule-based branching, and image segmentation by AI, resulting in improved analysis accuracy, reduction of misdiagnosis risk, significant improvement in analysis speed, efficiency of data management, and improved diagnostic accuracy through integrated analysis of multiple examination results, thereby improving computer technology itself. Specific application fields include remote image diagnosis support, rapid diagnosis in emergency medical settings, chronic disease management, and automatic anomaly detection in health checkup centers.

The proposal unit can identify a hospital based on specialist data. For example, the proposal unit identifies a hospital based on specialist data. For example, the proposal unit identifies a hospital based on specialist qualification information and treatment records. The proposal unit can also evaluate hospitals based on specialist data. For example, the proposal unit evaluates hospitals based on specialist treatment records and patient evaluations. The proposal unit can also set hospital selection criteria based on specialist data. For example, the proposal unit sets hospital selection criteria based on specialist qualification information and treatment records. By identifying hospitals based on specialist data, appropriate hospital proposals become possible. Some or all of the above-described processing in the proposal unit may be performed using AI or without using AI. For example, the proposal unit can input specialist data into AI and have the AI perform hospital identification. Specifically, the proposal unit refers to a specialist database (e.g., physician ID, specialty, qualification information, number of treatment records, patient evaluation score, hospital location, department, accessibility, etc. as structured data) and inputs these data as input vectors (e.g., 1×P dimensions, where P is the number of items) into an AI model. The proposal unit uses an AI model (e.g., gradient boosting decision tree, random forest, or Transformer-based large language model) to integratively analyze the input specialist data and user symptom/examination result data and generates as output an optimal hospital candidate list (e.g., hospital ID list with recommendation scores), recommendation order, and recommendation reasons (e.g., many treatment records, high patient evaluation, good accessibility, etc.). Input examples to the AI model include specialist qualification information (gastroenterology specialist, 200 treatment records, patient evaluation 4.8), hospital location (Tokyo), department (gastroenterology), accessibility (5 minutes walk from station), etc. Output examples from the AI model include recommended hospital list (Hospital A 0.92, Hospital B 0.85, Hospital C 0.80), recommendation reasons (many treatment records, high patient evaluation), etc. These outputs are used for hospital list display in the display unit and for determining the proposal order to the user. The proposal unit can also automatically adjust hospital selection criteria (e.g., emphasis on treatment records, patient evaluation, accessibility, etc.) according to user preferences and symptoms. These processes, unlike conventional human hospital referrals or simple database searches, realize unconventional techniques such as multidimensional data analysis, scoring, rule-based branching, and user suitability estimation by AI, resulting in improved proposal accuracy, increased user satisfaction, significant improvement in proposal speed, efficiency of data management, and automation of individualized optimal proposals, thereby improving computer technology itself. Specific application fields include specialist referral in telemedicine platforms, rapid hospital selection in emergency medical settings, specialist matching for chronic disease patients, and medical institution recommendation in corporate health management support services.

The estimation unit can estimate a disease name by analyzing physical conditions such as body temperature, nausea, headache, and the location and degree of pain. For example, the estimation unit analyzes body temperature. For example, if the body temperature is high, the estimation unit estimates a disease accompanied by fever. The estimation unit can also analyze nausea. For example, if nausea is present, the estimation unit estimates a gastrointestinal disease. The estimation unit can also analyze headache. For example, if headache is present, the estimation unit estimates a neurological disease. The estimation unit can also analyze the location and degree of pain. For example, if the pain is concentrated in a specific location, the estimation unit estimates a disease related to that location. By analyzing physical conditions and the location and degree of pain, more accurate disease name estimation becomes possible. Some or all of the above-described processing in the estimation unit may be performed using generative AI or without using generative AI. For example, the estimation unit can input physical condition information such as body temperature, nausea, headache, etc. into generative AI and have the generative AI perform disease name estimation. Specifically, the estimation unit receives physical condition information input by the user (e.g., body temperature 36.8° C., nausea present, headache absent, pain location: lower right abdomen, pain degree: 7, etc.) as numerical vectors of 1×N dimensions or categorical data. The estimation unit normalizes (e.g., Z-score normalization), standardizes, imputes missing values (e.g., median imputation), and detects outliers (e.g., IQR method or Z-score judgment) in the preprocessing unit and formats the data as input tensors for estimation. The estimation unit, for example, uses a multilayer perceptron, convolutional neural network, or Transformer-based large language model to input the physical condition information vector and generates as output a label list of candidate disease names (e.g., gastroenteritis, migraine, influenza, etc.) and probability scores for each disease name (e.g., gastroenteritis 0.65, migraine 0.20, influenza 0.10, etc.). Inside the AI model, techniques such as ReLU activation function, batch normalization, and dropout are combined to perform inference using trained weights. Input examples to the AI model include vector data such as body temperature 36.8° C., nausea present, headache absent, pain location: lower right abdomen, pain degree: 7, etc. Output examples from the AI model include candidate disease name list (gastroenteritis 0.65, migraine 0.20, influenza 0.10), outlier flag (outlier flag 1 if body temperature is 39° C.), etc. The estimation unit sends requests for hospital selection and treatment policy determination to subsequent display and proposal units based on the output candidate disease names and probability scores. These processes, unlike conventional human interviews and rule-of-thumb judgments, realize unconventional techniques such as feature extraction in high-dimensional vector space, probabilistic inference, and rule-based branching by AI, resulting in improved estimation accuracy, reduction of misdiagnosis risk, significant improvement in processing speed, and efficiency of data management, thereby improving computer technology itself. Specific application fields include triage support in emergency medical settings, telemedicine platforms, chronic disease management systems, and corporate health management support services.

The display unit can display a list of hospitals to be visited based on the estimated disease name. For example, the display unit displays a list of hospitals to be visited based on the estimated disease name. For example, the display unit selects a hospital by considering the specialty, distance, and evaluation of the hospital based on the estimated disease name. The display unit can also visually display the list of hospitals. For example, the display unit displays information such as the hospital name, address, telephone number, and department. The display unit can also display hospital evaluation and review information. For example, the display unit displays hospital evaluation scores and patient review information. By displaying a list of hospitals to be visited based on the estimated disease name, the user can select an appropriate hospital. Some or all of the above-described processing in the display unit may be performed using AI or without using AI. For example, the display unit can input a list of hospitals to be visited based on the estimated disease name into generative AI and have the generative AI perform the display of the hospital list. Specifically, the display unit receives as input the candidate disease name list and probability scores (e.g., gastroenteritis 0.65, migraine 0.20, influenza 0.10, etc.) from the estimation unit. The display unit obtains structured data such as specialty (e.g., gastroenterology, neurology, etc.), distance to hospital, past treatment records, patient evaluation scores, hospital location, department, and accessibility from the database for each disease name and inputs these as 1×P dimensional input vectors into the AI model. The AI model (e.g., gradient boosting decision tree, random forest, Transformer-based large language model, etc.) integratively analyzes the input disease name and hospital data and generates as output an optimal hospital list (e.g., hospital ID list with recommendation scores), recommendation order, and recommendation reasons (e.g., many treatment records, high patient evaluation, good accessibility, etc.). Input examples to the AI model include disease name “gastroenteritis,” specialty “gastroenterology,” distance 2.5 km, patient evaluation 4.8, 200 treatment records, etc. Output examples from the AI model include recommended hospital list (Hospital A 0.92, Hospital B 0.85, Hospital C 0.80), recommendation reasons (many treatment records, high patient evaluation), etc. The display unit visually displays information such as hospital name, address, telephone number, department, evaluation score, and review information on the user interface based on these outputs. Furthermore, the display unit can automatically adjust the display method (e.g., simple display, detailed display, ranking display, etc.) according to the user's emotion and situation. These processes, unlike conventional human hospital referrals or simple database searches, realize unconventional techniques such as multidimensional data analysis, scoring, rule-based branching, and user suitability estimation by AI, resulting in improved proposal accuracy, increased user satisfaction, significant improvement in proposal speed, efficiency of data management, and automation of individualized optimal proposals, thereby improving computer technology itself. Specific application fields include specialist referral in telemedicine platforms, rapid hospital selection in emergency medical settings, specialist matching for chronic disease patients, and medical institution recommendation in corporate health management support services.

The receiving unit can estimate the user's emotion and change the input method for physical condition information based on the estimated emotion of the user. For example, the receiving unit estimates the user's emotion. For example, the receiving unit estimates the user's emotion using facial recognition technology. The receiving unit can also estimate the user's emotion using voice analysis technology. For example, the receiving unit analyzes the tone and speed of the user's voice to estimate emotion. The receiving unit can change the input method for physical condition information based on the user's emotion. For example, if the user is feeling stressed, the receiving unit provides a simple interface and minimizes the input steps. If the user is relaxed, the receiving unit provides detailed input options and proposes customizable input methods. If the user is in a hurry, the receiving unit prioritizes voice input to enable quick entry of physical condition information. By adjusting the input method for physical condition information according to the user's emotion, more appropriate input becomes possible. Emotion estimation is realized using, for example, an emotion engine or generative AI with emotion estimation functions. Generative AI may be text generation AI (e.g., LLM) or multimodal generative AI, but is not limited to such examples. Some or all of the above-described processing in the receiving unit may be performed using AI or without using AI. For example, the receiving unit can input the user's emotion data into generative AI and have the generative AI perform emotion estimation. Specifically, the receiving unit simultaneously acquires facial images (e.g., RGB images, 224×224 pixels), voice waveform data (e.g., 16 kHz sampling, 5-second voice clip), and text data at the time of input (e.g., short sentences or word sequences entered by the user) for emotion estimation and inputs these as multimodal input tensors into the AI model. For image data, the receiving unit applies convolutional neural networks (e.g., ResNet-based); for voice data, after spectrogram conversion, convolutional neural networks or recurrent neural networks (e.g., LSTM); and for text data, Transformer-based large language models. An attention fusion layer is provided to integrate these feature extraction results, and finally, emotion labels (e.g., stress, relaxation, anxiety, hurry, etc.) and probability distributions for each emotion (e.g., stress 0.70, relaxation 0.10, anxiety 0.15, hurry 0.05) are output. Input examples to the AI model include a smiling face image+text such as “I am not feeling well today”+a calm voice clip, or a tense face image+text such as “I'm in a hurry”+a fast-talking voice clip. Output examples from the AI model include emotion label “stress” probability 0.80, “relaxation” probability 0.10, “hurry” probability 0.10, or “relaxation” probability 0.85, “anxiety” probability 0.05, etc. Based on these emotion estimation results, the receiving unit automatically switches the layout of the user interface, the number of input items, and the input method (e.g., voice input, selection input, detailed input, etc.). For example, if stress is high, a simple UI with limited choices is presented; if relaxed, detailed input items are presented; if in a hurry, voice input is prioritized, etc. These processes, unlike conventional subjective judgment by human operators or uniform UI provision, realize automatic determination of optimal input methods by AI analyzing high-dimensional multimodal data and combining probabilistic inference and rule-based branching, resulting in improved input accuracy, reduced user stress, reduced input errors, improved input completion rate, and improved overall system usability, thereby improving computer technology itself. Specific application fields include remote medical reception systems, wearable device-linked health record apps, corporate health management platforms, and triage reception terminals in emergency medical settings.

The receiving unit can analyze the user's past physical condition information and provide an appropriate input format. For example, the receiving unit analyzes the user's past physical condition information. For example, the receiving unit automatically displays frequently entered items based on the user's past physical condition information. The receiving unit can also prioritize input items related to specific symptoms based on the user's past physical condition information. For example, the receiving unit analyzes the user's past physical condition information and provides shortcuts to reduce input effort. By analyzing past physical condition information, input effort can be reduced and efficient input becomes possible. Some or all of the above-described processing in the receiving unit may be performed using AI or without using AI. For example, the receiving unit can input the user's past physical condition information into AI and have the AI provide the input format. Specifically, the receiving unit maintains a database of physical condition information recorded in time series for each user (e.g., structured data including date, body temperature, blood pressure, heart rate, symptom label, pain location, pain degree, etc., as an array of 1×N dimensional vectors) and inputs these data into the AI model. The AI model uses recurrent neural networks (e.g., LSTM, GRU) or time-series Transformer models to learn past input patterns, frequently entered items, and symptom transition trends. Input examples to the AI model include an array of physical condition vectors for the past 30 days (e.g., body temperature 36.8, 36.7, 37.0 . . . , headache present/absent, pain location: lower right abdomen/left shoulder, etc.), and items frequently entered in the past (e.g., “nausea” or “abdominal pain” entered every time). Output examples from the AI model include input UI customization instructions such as “prioritize display of abdominal pain item today,” “expand headache input field by default,” “display pain degree shortcut,” or layout optimization instructions such as “display items entered three times in the past seven days at the top.” Based on these AI outputs, the receiving unit automatically adjusts the order of input form items, display presence, and shortcut buttons for each user. Furthermore, if a specific symptom persists, related additional items (e.g., “bowel movement,” “appetite,” etc. if abdominal pain continues) are automatically displayed, realizing dynamic UI generation. These processes, unlike conventional static input forms or manual customization by human operators, realize automatic generation of optimal input formats by AI combining time-series data analysis, pattern recognition, and rule-based branching, resulting in significant improvement in input efficiency, reduction of input errors, reduction of user burden, promotion of continuous health recording, and improvement of overall system usability, thereby improving computer technology itself. Specific application fields include chronic disease management apps, remote health recording systems, corporate health management support services, and wearable device-linked health management platforms.

The receiving unit can perform filtering based on the user's lifestyle habits and medical history when inputting physical condition information. For example, the receiving unit considers the user's lifestyle habits. For example, the receiving unit prioritizes input of relevant physical condition information based on the user's smoking or drinking habits. The receiving unit can also consider the user's medical history. For example, the receiving unit filters relevant physical condition information based on the user's past medical history. The receiving unit can also customize input items based on the user's lifestyle habits and medical history. For example, the receiving unit automatically adjusts input items based on the user's lifestyle habits and medical history. By performing filtering based on lifestyle habits and medical history, more relevant physical condition information can be input. Some or all of the above-described processing in the receiving unit may be performed using AI or without using AI. For example, the receiving unit can input the user's lifestyle habits and medical history data into AI and have the AI perform filtering. Specifically, the receiving unit obtains lifestyle habit data recorded for each user (e.g., categorical data or numerical vectors such as smoking status, drinking frequency, exercise habits, dietary content, etc.) and medical history data (e.g., list of past diagnosed disease names, treatment history, surgical history, etc. as structured data) from the database and inputs these into the AI model. The AI model uses gradient boosting decision trees, random forests, or Transformer-based large language models to learn the relationship between lifestyle habits/medical history and physical condition information input items. Input examples to the AI model include vector data such as “smoker, drinks three times a week, previously diagnosed with hypertension,” “no exercise habit, history of diabetes,” etc. Output examples from the AI model include input item customization instructions such as “prioritize display of respiratory symptom input field for smokers,” “add blood glucose and foot numbness input fields for those with a history of diabetes,” “automatically expand liver function-related items for those with drinking habits,” etc. Based on these AI outputs, the receiving unit automatically adjusts the order of input form item display, required items, and additional items for each user. Furthermore, if changes in lifestyle habits or medical history are detected, dynamic reconfiguration of input items and alert display can also be performed. These processes, unlike conventional uniform input forms or manual customization by human operators, realize automatic presentation of optimal input items by AI combining multidimensional structured data analysis, rule-based branching, and dynamic UI generation, resulting in improved input accuracy, increased comprehensiveness of relevant information, reduction of user burden, early detection of health risks, and improvement of overall system usability, thereby improving computer technology itself. Specific application fields include lifestyle disease management systems, corporate health management support, home monitoring for chronic disease patients, and wearable device-linked health record services.

The receiving unit can estimate the user's emotion and determine the order of physical condition information to be input based on the estimated emotion of the user. For example, the receiving unit estimates the user's emotion. For example, the receiving unit estimates the user's emotion using facial recognition technology. The receiving unit can also estimate the user's emotion using voice analysis technology. For example, the receiving unit analyzes the tone and speed of the user's voice to estimate emotion. The receiving unit can determine the order of physical condition information to be input based on the user's emotion. For example, if the user is feeling anxious, the receiving unit prompts the user to input important physical condition information first. If the user is relaxed, the receiving unit proposes input of detailed physical condition information. If the user is in a hurry, the receiving unit prioritizes input of the most important physical condition information. By determining the priority of physical condition information according to the user's emotion, important information can be input first. Emotion estimation is realized using, for example, an emotion engine or generative AI with emotion estimation functions. Generative AI may be text generation AI (e.g., LLM) or multimodal generative AI, but is not limited to such examples. Some or all of the above-described processing in the receiving unit may be performed using AI or without using AI. For example, the receiving unit can input the user's emotion data into generative AI and have the generative AI perform emotion estimation. Specifically, the receiving unit inputs the user's facial image (e.g., 224×224 pixel RGB image), voice data (e.g., 16 kHz sampling, 5-second voice clip), and text data at the time of input (e.g., short sentences or word sequences) as multimodal input tensors into the AI model, and outputs emotion labels (e.g., anxiety, relaxation, hurry, etc.) and probability distributions for each emotion (e.g., anxiety 0.70, relaxation 0.20, hurry 0.10). Input examples to the AI model include a tense face image+text such as “I'm in a hurry”+a fast-talking voice clip, or a calm face image+text such as “I want to input slowly”+a calm voice clip. Output examples from the AI model include emotion label “anxiety” probability 0.80, “relaxation” probability 0.10, “hurry” probability 0.10, or “relaxation” probability 0.85, “anxiety” probability 0.05, etc. Based on these emotion estimation results, the receiving unit automatically determines the priority of physical condition information input items. For example, if anxiety is high, input fields for serious symptoms such as “fever” and “dyspnea” are displayed at the top; if relaxed, additional detailed items such as “sleep” and “appetite” are displayed; if in a hurry, only minimal items such as “body temperature” and “pain location” are displayed first, etc. These processes, unlike conventional uniform input order or subjective judgment by human operators, realize automatic determination of optimal input order by AI combining multimodal data analysis, probabilistic inference, and rule-based branching, resulting in improved input efficiency, prevention of omission of important information, reduced user stress, improved input completion rate, and improved overall system usability, thereby improving computer technology itself. Specific application fields include emergency medical reception terminals, remote health recording systems, chronic disease management apps, and corporate health management support services.

The receiving unit can consider the user's geographic location information when inputting physical condition information and prioritize the input of highly relevant information. For example, the receiving unit considers the user's geographic location information. For example, the receiving unit prioritizes input of physical condition information related to region-specific diseases or symptoms based on the user's current location. The receiving unit can also refer to the examination history at nearby medical institutions based on the user's geographic location information. For example, the receiving unit inputs physical condition information related to the climate or environment of the region based on the user's geographic location information. By considering geographic location information, information related to region-specific diseases or symptoms can be prioritized for input. Some or all of the above-described processing in the receiving unit may be performed using AI or without using AI. For example, the receiving unit can input the user's geographic location information into AI and have the AI perform input of highly relevant information. Specifically, the receiving unit inputs latitude and longitude information (e.g., 35.6895, 139.6917) obtained from the user's device GPS or IP address, region codes linked to location information, and past examination history data (e.g., examination history at nearby medical institutions, region-specific epidemic disease database) into the AI model. The AI model uses geospatial information analysis algorithms (e.g., spatial clustering, geographic feature extraction) and large language models to learn the relationship between region-specific disease risks or environmental factors (e.g., pollen dispersion, temperature, humidity, epidemic status, etc.) and physical condition information input items. Input examples to the AI model include geographic and seasonal information vectors such as “Shinjuku, Tokyo, spring, pollen allergy epidemic,” “Osaka, summer, high heatstroke risk,” etc. Output examples from the AI model include input item customization instructions such as “prioritize display of pollen allergy-related items,” “add heatstroke risk items,” “display fever and cough input fields at the top during influenza epidemic,” etc. Based on these AI outputs, the receiving unit automatically adjusts the order of input form item display, required items, and additional items according to the user's current location and regional characteristics. Furthermore, by linking with regional medical institution treatment records and epidemic disease databases, dynamic generation of input items tailored to region-specific health risks can also be realized. These processes, unlike conventional uniform input forms or manual reflection of regional information by human operators, realize automatic presentation of optimal input items by AI combining geospatial data analysis, rule-based branching, and dynamic UI generation, resulting in improved input accuracy, early detection of region-specific risks, reduction of user burden, regional optimization of health management, and improvement of overall system usability, thereby improving computer technology itself. Specific application fields include regional medical collaboration platforms, remote health recording systems, corporate health management support services, and disaster health management systems.

The receiving unit can analyze the user's social media activity when inputting physical condition information and input relevant information. For example, the receiving unit analyzes the user's social media activity. For example, the receiving unit extracts and inputs recent physical condition information from the user's social media posts. The receiving unit can also automatically input keywords related to physical condition by analyzing the user's social media activity. For example, the receiving unit supplements physical condition information based on the user's social media activity history. By analyzing social media activity, information related to physical condition can be supplemented. Some or all of the above-described processing in the receiving unit may be performed using AI or without using AI. For example, the receiving unit can input the user's social media activity data into AI and have the AI perform input of relevant information. Specifically, the receiving unit inputs social media post data obtained within the permitted scope by the user (e.g., text posts, images, post date/time, hashtags, etc. as structured data) into the AI model. The AI model uses natural language processing algorithms (e.g., Transformer-based large language models) and image analysis models (e.g., CNN) to extract keywords related to physical condition (e.g., “headache,” “fever,” “fatigue,” etc.), symptom expressions, post frequency, and time-series patterns from post content. Input examples to the AI model include text posts such as “My headache has been severe since yesterday,” “I have a fever today,” “#not feeling well,” or selfie images with poor complexion. Output examples from the AI model include physical condition information supplement data such as “headache present,” “fever present,” “poor physical condition flag 1,” or time-series analysis results such as “three ‘fatigue’ posts in the past seven days.” Based on these AI outputs, the receiving unit automatically adds supplement items to the user's physical condition information input form or presents input candidates. Furthermore, by time-series analysis of post content, detection of worsening physical condition or persistence of symptoms, alert display, and promotion of additional input can also be realized. These processes, unlike conventional self-reporting by users or manual supplementation by human operators, realize automatic supplementation of physical condition information by AI combining natural language processing, image analysis, and time-series analysis, resulting in improved input accuracy, reduced input burden, early detection of symptoms, automation of health management, and improvement of overall system usability, thereby improving computer technology itself. Specific application fields include remote health recording systems, chronic disease management apps, corporate health management support services, and wearable device-linked health management platforms.

The estimation unit can estimate the user's emotion and change the disease name estimation method based on the estimated emotion of the user. For example, the estimation unit estimates the user's emotion. For example, the estimation unit estimates the user's emotion using facial recognition technology. The estimation unit can also estimate the user's emotion using voice analysis technology. For example, the estimation unit analyzes the tone and speed of the user's voice to estimate emotion. The estimation unit can change the disease name estimation method based on the user's emotion. For example, if the user is feeling anxious, the estimation unit provides disease name estimation results including detailed explanations. If the user is relaxed, the estimation unit provides concise disease name estimation results. If the user is in a hurry, the estimation unit quickly estimates a disease name and displays the result. By adjusting the disease name estimation method according to the user's emotion, more appropriate estimation results can be provided. Emotion estimation is realized using, for example, an emotion engine or generative AI with emotion estimation functions. Generative AI may be text generation AI (e.g., LLM) or multimodal generative AI, but is not limited to such examples. Some or all of the above-described processing in the estimation unit may be performed using AI or without using AI. For example, the estimation unit can input the user's emotion data into generative AI and have the generative AI perform emotion estimation. Specifically, the estimation unit simultaneously acquires facial images (e.g., 224×224 pixel RGB images), voice waveform data (e.g., 16 kHz sampling, 5-second voice clip), and text data at the time of input (e.g., short sentences or word sequences entered by the user) for emotion estimation and inputs these as multimodal input tensors into the AI model. For image data, the estimation unit applies convolutional neural networks (e.g., ResNet-based); for voice data, after spectrogram conversion, convolutional neural networks or recurrent neural networks (e.g., LSTM); and for text data, Transformer-based large language models. An attention fusion layer is provided to integrate these feature extraction results, and finally, emotion labels (e.g., stress, relaxation, anxiety, hurry, etc.) and probability distributions for each emotion (e.g., stress 0.70, relaxation 0.10, anxiety 0.15, hurry 0.05) are output. Input examples to the AI model include a smiling face image+text such as “I am not feeling well today”+a calm voice clip, or a tense face image+text such as “I'm in a hurry”+a fast-talking voice clip. Output examples from the AI model include emotion label “stress” probability 0.80, “relaxation” probability 0.10, “hurry” probability 0.10, or “relaxation” probability 0.85, “anxiety” probability 0.05, etc. Based on these emotion estimation results, the estimation unit automatically switches the branching logic of the disease name estimation algorithm. For example, if anxiety is high, the estimation unit utilizes the explanation generation function of the large language model to generate disease name estimation results with detailed descriptions of the estimation basis, relationship to symptoms, and necessity of additional examinations. If relaxed, the estimation unit displays only the main candidate disease names and their probability scores concisely. If in a hurry, the estimation unit omits part of the inference pipeline and immediately displays only the most probable disease name, thereby speeding up the estimation process. These branches are controlled by threshold judgment based on the output probability of the AI model and rule-based branching logic. Furthermore, the estimation unit can link emotion estimation results to subsequent display and proposal units to optimize the overall user experience. These processes, unlike conventional uniform disease name estimation or subjective judgment by human operators, realize automatic determination of optimal estimation methods by AI combining multimodal data analysis, probabilistic inference, and rule-based branching, resulting in improved estimation accuracy, increased user satisfaction, reduction of misdiagnosis risk, and improvement of overall system usability, thereby improving computer technology itself. Specific application fields include triage support in emergency medical settings, telemedicine platforms, chronic disease management systems, and corporate health management support services.

The estimation unit can adjust the level of detail of estimation based on the importance of the physical condition information when estimating a disease name. For example, the estimation unit evaluates the importance of physical condition information. For example, the estimation unit evaluates the importance of physical condition information based on the severity of symptoms and frequency of occurrence. The estimation unit can adjust the level of detail of estimation based on the importance of physical condition information. For example, the estimation unit provides detailed disease name estimation results based on important physical condition information. The estimation unit can also provide concise disease name estimation results based on minor physical condition information. The estimation unit adjusts the level of detail of estimation according to the importance of physical condition information. By adjusting the level of detail of estimation according to the importance of physical condition information, more accurate estimation results can be provided. Some or all of the above-described processing in the estimation unit may be performed using AI or without using AI. For example, the estimation unit can input the importance of physical condition information into AI and have the AI adjust the level of detail of estimation. Specifically, the estimation unit receives physical condition information input by the user (e.g., body temperature 36.8° C., blood pressure 120/80 mmHg, heart rate 78 bpm, nausea present, headache absent, pain location: lower right abdomen, pain degree: 7, etc.) as numerical vectors of 1×N dimensions or categorical data. The estimation unit normalizes (e.g., Z-score normalization), standardizes, imputes missing values (e.g., median imputation), and detects outliers (e.g., IQR method or Z-score judgment) in the preprocessing unit and formats the data as input tensors for estimation. The estimation unit sets weighting parameters for each physical condition information item and calculates importance scores based on indicators such as severity and frequency of occurrence. The AI model (e.g., multilayer perceptron, Transformer-based large language model, etc.) combines the input physical condition information vector and importance scores for inference and generates as output a candidate disease name list (e.g., gastroenteritis 0.65, migraine 0.20, influenza 0.10, etc.) and the level of detail of explanation for each disease name (e.g., detailed explanation, concise explanation, key points only, etc.). Input examples to the AI model include vector data such as body temperature 39.0° C. (high severity), nausea present (high frequency), pain degree 2 (minor), etc. Output examples from the AI model include candidate disease name “influenza” with detailed explanation, “gastroenteritis” with concise explanation, “migraine” with key points only, etc. For physical condition information with high importance scores, the estimation unit generates estimation results with detailed descriptions of the estimation basis, necessity of additional examinations, and treatment policy; for low importance, only the main candidate disease names and probability scores are displayed concisely. These branches are controlled by threshold judgment based on the output probability and weighting parameters of the AI model and rule-based branching logic. Furthermore, the estimation unit can link importance evaluation results to subsequent display and proposal units to optimize the overall user experience. These processes, unlike conventional uniform disease name estimation or subjective judgment by human operators, realize automatic determination of optimal estimation detail by AI combining feature extraction, weighting, probabilistic inference, and rule-based branching in high-dimensional vector space, resulting in improved estimation accuracy, increased user satisfaction, reduction of misdiagnosis risk, and improvement of overall system usability, thereby improving computer technology itself. Specific application fields include triage support in emergency medical settings, telemedicine platforms, chronic disease management systems, and corporate health management support services.

The estimation unit can apply different estimation algorithms according to the category of physical condition information when estimating a disease name. For example, the estimation unit classifies the category of physical condition information. For example, the estimation unit classifies physical condition information into categories such as internal medicine, surgery, psychiatry, etc. The estimation unit can apply different estimation algorithms according to the category of physical condition information. For example, the estimation unit applies standard estimation algorithms to general symptoms such as body temperature and nausea. The estimation unit can also apply specialized estimation algorithms to pain in specific locations or degrees. The estimation unit selects the optimal estimation algorithm according to the category of physical condition information. By applying the optimal estimation algorithm according to the category of physical condition information, more accurate estimation results can be provided. Some or all of the above-described processing in the estimation unit may be performed using AI or without using AI. For example, the estimation unit can input the category of physical condition information into AI and have the AI apply the estimation algorithm. Specifically, the estimation unit receives physical condition information input by the user (e.g., body temperature 36.8° C., nausea present, headache absent, pain location: lower right abdomen, pain degree: 7, etc.) as numerical vectors of 1×N dimensions or categorical data. The estimation unit normalizes, standardizes, and classifies categories (e.g., internal medicine, surgery, psychiatry, dermatology, etc.) in the preprocessing unit and selects different estimation algorithms for each category. For example, for internal medicine symptoms (e.g., fever, nausea, diarrhea, etc.), multilayer perceptron or gradient boosting decision tree is applied; for surgical symptoms (e.g., localized pain, trauma, etc.), convolutional neural network or rule-based inference engine is applied; for psychiatric symptoms (e.g., anxiety, depression, etc.), large language model or time-series analysis model (e.g., LSTM) is applied. Input examples to the AI model include vector data such as body temperature 38.5° C., nausea present (internal medicine), right lower abdominal pain, pain degree 8 (surgery), depressed mood, insomnia (psychiatry), etc. Output examples from the AI model include internal medicine: “gastroenteritis 0.65, influenza 0.20”; surgery: “appendicitis 0.80, intestinal obstruction 0.10”; psychiatry: “depression 0.70, anxiety disorder 0.20,” etc. Based on the category classification results, the estimation unit automatically switches the branching logic of the estimation algorithm and performs inference with models optimized for each category. Furthermore, if symptoms span multiple categories, multitask learning models or multimodal fusion models can be applied for comprehensive disease name estimation. These processes, unlike conventional uniform disease name estimation or subjective judgment by human operators, realize automatic determination of optimal estimation methods by AI combining feature extraction, category classification, algorithm branching, and multimodal inference in high-dimensional vector space, resulting in improved estimation accuracy, reduction of misdiagnosis risk, increased system flexibility, and improved usability, thereby improving computer technology itself. Specific application fields include general medical support systems, triage support in emergency medical settings, telemedicine platforms, and chronic disease management systems.

The estimation unit can estimate the user's emotion and change the order of displaying disease name estimation results based on the estimated emotion of the user. For example, the estimation unit estimates the user's emotion. For example, the estimation unit estimates the user's emotion using facial recognition technology. The estimation unit can also estimate the user's emotion using voice analysis technology. For example, the estimation unit analyzes the tone and speed of the user's voice to estimate emotion. The estimation unit can change the order of displaying disease name estimation results based on the user's emotion. For example, if the user is feeling anxious, the estimation unit displays the most probable disease name first. If the user is relaxed, the estimation unit adjusts the order according to the probability of disease names. If the user is in a hurry, the estimation unit quickly displays the disease name estimation results. By adjusting the order of displaying disease name estimation results according to the user's emotion, more appropriate information can be provided. Emotion estimation is realized using, for example, an emotion engine or generative AI with emotion estimation functions. Generative AI may be text generation AI (e.g., LLM) or multimodal generative AI, but is not limited to such examples. Some or all of the above-described processing in the estimation unit may be performed using AI or without using AI. For example, the estimation unit can input the user's emotion data into generative AI and have the generative AI perform emotion estimation. Specifically, the estimation unit simultaneously acquires facial images (e.g., 224×224 pixel RGB images), voice waveform data (e.g., 16 kHz sampling, 5-second voice clip), and text data at the time of input (e.g., short sentences or word sequences entered by the user) for emotion estimation and inputs these as multimodal input tensors into the AI model. For image data, the estimation unit applies convolutional neural networks (e.g., ResNet-based); for voice data, after spectrogram conversion, convolutional neural networks or recurrent neural networks (e.g., LSTM); and for text data, Transformer-based large language models. An attention fusion layer is provided to integrate these feature extraction results, and finally, emotion labels (e.g., stress, relaxation, anxiety, hurry, etc.) and probability distributions for each emotion (e.g., stress 0.70, relaxation 0.10, anxiety 0.15, hurry 0.05) are output. Input examples to the AI model include a tense face image+text such as “I'm in a hurry”+a fast-talking voice clip, or a calm face image+text such as “I want to input slowly”+a calm voice clip. Output examples from the AI model include emotion label “anxiety” probability 0.80, “relaxation” probability 0.10, “hurry” probability 0.10, or “relaxation” probability 0.85, “anxiety” probability 0.05, etc. Based on these emotion estimation results, the estimation unit automatically determines the order of displaying disease name estimation results. For example, if anxiety is high, the estimation unit displays the candidate disease name with the highest probability score at the top and adds a detailed explanation. If relaxed, the estimation unit arranges candidate disease names in order of probability or relevance and adds concise explanations. If in a hurry, only the most probable disease name is displayed immediately and other candidates are omitted, thereby speeding up the display process. These branches are controlled by threshold judgment based on the output probability of the AI model and rule-based branching logic. Furthermore, the estimation unit can link emotion estimation results to subsequent display and proposal units to optimize the overall user experience. These processes, unlike conventional uniform disease name display or subjective judgment by human operators, realize automatic determination of optimal display order by AI combining multimodal data analysis, probabilistic inference, and rule-based branching, resulting in improved appropriateness of information provision, increased user satisfaction, reduction of misdiagnosis risk, and improvement of overall system usability, thereby improving computer technology itself. Specific application fields include triage support in emergency medical settings, telemedicine platforms, chronic disease management systems, and corporate health management support services.

The estimation unit can determine the priority of estimation based on the submission timing of the physical condition information when estimating a disease name. For example, the estimation unit evaluates the submission timing of physical condition information. For example, the estimation unit evaluates the submission timing based on the submission date/time and frequency of physical condition information. The estimation unit can determine the priority of estimation based on the submission timing of physical condition information. For example, the estimation unit prioritizes disease name estimation based on recently submitted physical condition information. The estimation unit can also adjust the priority of estimation based on past physical condition information. The estimation unit determines the priority of estimation according to the submission timing of physical condition information. By determining the priority of estimation according to the submission timing of physical condition information, more rapid estimation results can be provided. Some or all of the above-described processing in the estimation unit may be performed using AI or without using AI. For example, the estimation unit can input the submission timing of physical condition information into AI and have the AI determine the priority of estimation. Specifically, the estimation unit stores physical condition information input by the user together with submission date/time (e.g., Jun. 1, 2024, 10:23:00) and submission frequency (e.g., once a day, three times a week, etc.) as structured data with timestamps (e.g., 1×N dimensional vector+timestamp array) in a database. The estimation unit performs time-series normalization (e.g., standardization of submission intervals, encoding of time), aggregation of submission frequency, and weighting of submission timing (e.g., higher weight for recent data) in the preprocessing unit and formats the data as input tensors for estimation. The estimation unit uses an AI model such as recurrent neural network (LSTM or GRU), time-series Transformer, or a combination of multilayer perceptron and time-series feature extraction module to input the physical condition information vector and submission timing information simultaneously and generates as output a candidate disease name list (e.g., gastroenteritis 0.65, migraine 0.20, influenza 0.10, etc.) and priority scores for each disease name (e.g., gastroenteritis 0.90, migraine 0.60, etc.). Input examples to the AI model include body temperature 38.5° C., submission date/time 2024/06/01 10:23, submission frequency once a day, or body temperature 36.8° C., submission date/time 2024/05/25 08:00, submission frequency once a week, etc. Output examples from the AI model include candidate disease name “influenza” priority 0.95, “gastroenteritis” priority 0.60, “migraine” priority 0.30, etc. Based on the output priority scores, the estimation unit prioritizes disease name estimation based on recent physical condition information, and past physical condition information is used as supplementary reference information with adjusted weights in the estimation algorithm. Furthermore, if submission timing is continuous or submission frequency is high, the estimation unit detects time-series changes in symptoms or signs of acute onset and automatically adjusts the urgency and level of detail of estimation results. These branches are controlled by threshold judgment based on the output probability and submission timing weighting parameters of the AI model and rule-based branching logic. Subsequent display and proposal units automatically optimize the order and urgency display of hospital selection and treatment policy proposals based on the priority disease name list received from the estimation unit. These processes, unlike conventional uniform disease name estimation or subjective judgment by human operators, realize automatic determination of optimal estimation priority by AI combining time-series data analysis, submission timing weighting, probabilistic inference, and rule-based branching, resulting in improved estimation accuracy, rapid response to emergency cases, increased user satisfaction, reduction of misdiagnosis risk, and improvement of overall system usability, thereby improving computer technology itself. Specific application fields include triage support in emergency medical settings, acute exacerbation detection in chronic disease management systems, emergency alert issuance in remote health recording systems, and early detection of health risks in corporate health management support services.

The estimation unit can adjust the order of estimation based on the relevance of the physical condition information when estimating a disease name. For example, the estimation unit evaluates the relevance of physical condition information. For example, the estimation unit evaluates the relevance of physical condition information based on commonalities of symptoms and patterns of onset. The estimation unit can adjust the order of estimation based on the relevance of physical condition information. For example, the estimation unit displays the most relevant disease name first based on the relevance of physical condition information. The estimation unit can also adjust the order of estimation results according to the relevance of physical condition information. The estimation unit analyzes the relevance of physical condition information and optimizes the order of estimation. By adjusting the order of estimation based on the relevance of physical condition information, more relevant information can be provided. Some or all of the above-described processing in the estimation unit may be performed using AI or without using AI. For example, the estimation unit can input the relevance of physical condition information into AI and have the AI adjust the order of estimation. Specifically, the estimation unit receives physical condition information input by the user (e.g., body temperature 38.2° C., headache present, cough present, abdominal pain absent, pain location: lower right abdomen, pain degree: 5, etc.) as numerical vectors of 1×N dimensions or categorical data. The estimation unit normalizes (e.g., Z-score normalization), standardizes, imputes missing values (e.g., median imputation), and detects outliers (e.g., IQR method or Z-score judgment) in the preprocessing unit and formats the data as input tensors for estimation. To evaluate the relevance among physical condition information, the estimation unit extracts features such as co-occurrence frequency of symptoms, time-series patterns of onset, correlation coefficients with past diagnosis history, and symptom clustering results. The AI model (e.g., graph neural network, Transformer-based large language model, or multilayer perceptron) inputs these features and calculates a relevance score matrix among physical condition information (e.g., relevance between each symptom pair from 0.0 to 1.0). Input examples to the AI model include body temperature 38.2° C., headache present, cough present, abdominal pain absent, pain location: lower right abdomen, pain degree: 5, past 7-day symptom history vector, symptom co-occurrence matrix, etc. Output examples from the AI model include symptom relevance scores (e.g., headache-fever 0.85, cough-fever 0.80, abdominal pain-fever 0.10), highly relevant symptom groups (e.g., fever, headache, cough), candidate disease name list (influenza 0.70, cold 0.20, gastroenteritis 0.05), and display priority for each disease name (influenza 1st, cold 2nd, gastroenteritis 3rd), etc. Based on the highly relevant symptom groups, the estimation unit automatically determines the order of displaying candidate disease names. For example, if fever, headache, and cough are highly relevant, respiratory diseases (influenza, cold, etc.) are displayed at the top; if only abdominal pain is independent, gastrointestinal diseases are displayed lower. These branches are controlled by output scores from the AI model and rule-based branching logic. Furthermore, the estimation unit can link relevance evaluation results to subsequent display and proposal units and utilize them for optimization of hospital selection and treatment policy proposals. These processes, unlike conventional simple symptom lists or subjective judgment by human operators, realize automatic determination of optimal estimation order by AI combining feature extraction, symptom relevance analysis, graph structure analysis, probabilistic inference, and rule-based branching in high-dimensional vector space, resulting in improved estimation accuracy, increased comprehensiveness of related diseases, reduction of misdiagnosis risk, increased user satisfaction, and improvement of overall system usability, thereby improving computer technology itself. Specific application fields include general medical support systems, triage support in emergency medical settings, telemedicine platforms, chronic disease management systems, and corporate health management support services.

The display unit can estimate the user's emotion and change the display method of the list of hospitals based on the estimated emotion of the user. For example, the display unit estimates the user's emotion. For example, the display unit may use facial recognition technology to estimate the user's emotion. The display unit may also use voice analysis technology to estimate the user's emotion. For example, the display unit analyzes the tone and speed of the user's voice to estimate emotion. Furthermore, the display unit can change the display method of the list of hospitals based on the user's emotion. For example, if the user is feeling anxious, the display unit provides a simple and highly visible display method. If the user is relaxed, the display unit may provide a display method that includes detailed information. If the user is in a hurry, the display unit provides a display method that focuses on key points. By adjusting the display method of the list of hospitals according to the user's emotion, more appropriate information can be provided. Emotion estimation is realized, for example, by using an emotion engine or an emotion estimation function implemented by generative AI. Generative AI may be a text generation AI (e.g., LLM) or a multimodal generative AI, but is not limited to such examples. Some or all of the above-described processing in the display unit may be performed using AI or without using AI. For example, the display unit may input the user's emotion data to generative AI and have the generative AI perform emotion estimation. Specifically, the display unit simultaneously acquires facial images (e.g., 224×224 pixel RGB images), voice waveform data (e.g., 16 kHz sampling, 5-second audio clips), and text data at the time of input (e.g., short sentences or word sequences entered by the user) for emotion estimation, and inputs these as a multimodal input tensor to an AI model. For image data, the display unit applies a convolutional neural network (e.g., ResNet-based); for audio data, after spectrogram conversion, it applies a convolutional neural network or a recurrent neural network (e.g., LSTM); and for text data, it applies a Transformer-based large language model. A multimodal fusion layer (e.g., attention fusion layer) is provided to integrate these feature extraction results, and finally outputs emotion labels (e.g., stress, relaxation, anxiety, hurry, etc.) and probability distributions for each emotion (e.g., stress 0.70, relaxation 0.10, anxiety 0.15, hurry 0.05). Examples of input to the AI model include a stern facial image+text such as “I'm in a hurry”+a fast-spoken audio clip, or a calm facial image+text such as “I want to input slowly”+a calm voice audio clip. Examples of output from the AI model include emotion label “anxiety” probability 0.80, “relaxation” probability 0.10, “hurry” probability 0.10, or “relaxation” probability 0.85, “anxiety” probability 0.05, etc. The display unit automatically switches the display method of the list of hospitals based on these emotion estimation results. For example, if anxiety is high, only the hospital name, department, and phone number are displayed in large, simple text, and detailed information is collapsed. If the user is relaxed, detailed information such as hospital evaluation scores, reviews, treatment records, and access information is expanded and displayed. If the user is in a hurry, only the nearest hospitals or those available for immediate consultation are displayed in summary, and the UI layout and amount of information are dynamically adjusted. These branches are controlled by the output probabilities of the AI model or rule-based branching logic. Furthermore, the display unit can link the emotion estimation results to subsequent proposal units and estimation units to optimize the overall user experience. These processes differ from conventional uniform hospital list displays or subjective judgments by human operators, as AI automatically determines the optimal display method by combining multimodal data analysis, probabilistic inference, and rule-based branching, thereby improving the appropriateness of information provision, user satisfaction, completion rate of input, and overall system usability, which are improvements in computer technology itself. Specific application fields include specialist referral in telemedicine platforms, rapid hospital selection in emergency medical settings, specialist matching for chronic disease patients, and medical institution recommendation in corporate health management support services.

The display unit can adjust the level of detail of display based on the importance of the disease name when displaying the list of hospitals. For example, the display unit evaluates the importance of the disease name. For example, the display unit evaluates the importance of the disease name based on the severity of symptoms and the frequency of occurrence. The display unit can adjust the level of detail of display based on the importance of the disease name. For example, the display unit displays detailed hospital information based on important disease names. The display unit may also display concise hospital information based on minor disease names. The display unit adjusts the level of detail of display according to the importance of the disease name. By adjusting the level of detail of display according to the importance of the disease name, necessary information can be provided to the user. Some or all of the above-described processing in the display unit may be performed using AI or without using AI. For example, the display unit may input the importance of the disease name to AI and have the AI adjust the level of detail of display. Specifically, the display unit receives a list of candidate disease names and importance scores for each disease name (e.g., severity 0.9, frequency 0.7, etc.) from the estimation unit. The display unit obtains structured data such as specialty, distance to hospital, treatment records, patient evaluation scores, medical departments, and accessibility for each disease name from a database, and inputs these as a 1×P dimensional input vector to the AI model. The AI model (e.g., gradient boosting decision tree, random forest, Transformer-based large language model, etc.) integrally analyzes the input disease name, hospital data, and importance scores, and outputs the level of detail of display for each disease name (e.g., detailed display, concise display, key points only, etc.) and display item selection instructions (e.g., for serious disease names, display treatment records, specialist information, and urgency; for minor disease names, display only hospital name and department). Examples of input to the AI model include disease name “influenza” severity 0.9, disease name “migraine” severity 0.3, hospital evaluation 4.8, treatment records 200 cases, etc. Examples of output from the AI model include “influenza” detailed display, “migraine” concise display, “gastroenteritis” key points only, etc. The display unit automatically adjusts display items and information volume for each disease name on the user interface based on these AI outputs. For example, for serious disease names, the display unit displays details such as hospital emergency acceptance, specialist information, treatment records, and access information; for minor disease names, only the hospital name and department are displayed concisely. These branches are controlled by the output scores of the AI model or rule-based branching logic. Furthermore, the display unit can link the importance evaluation results to subsequent proposal units and estimation units to optimize the overall user experience. These processes differ from conventional uniform hospital information display or subjective judgment by human operators, as AI automatically determines the optimal level of detail of display by combining multidimensional data analysis, weighting, and rule-based branching, thereby improving the appropriateness of information provision, user satisfaction, reducing the risk of misdiagnosis, and improving overall system usability, which are improvements in computer technology itself. Specific application fields include triage support in emergency medical settings, telemedicine platforms, chronic disease management systems, and corporate health management support services.

The display unit can apply different display algorithms according to the category of the disease name when displaying the list of hospitals. For example, the display unit classifies the category of the disease name. For example, the display unit classifies disease names into categories such as internal medicine, surgery, psychiatry, etc. The display unit can apply different display algorithms according to the category of the disease name. For example, the display unit applies a standard display algorithm to general disease names. The display unit may also apply a specialized display algorithm to disease names related to specific specialties. The display unit selects the optimal display algorithm according to the category of the disease name. By applying the optimal display algorithm according to the category of the disease name, information that is easy for the user to view can be provided. Some or all of the above-described processing in the display unit may be performed using AI or without using AI. For example, the display unit may input the category of the disease name to AI and have the AI apply the display algorithm. Specifically, the display unit receives a list of candidate disease names and category information for each disease name (e.g., internal medicine, surgery, psychiatry, dermatology, etc.) from the estimation unit. The display unit obtains structured data such as specialty, distance to hospital, treatment records, patient evaluation scores, medical departments, and accessibility for each disease name from a database, and inputs these as a 1×P dimensional input vector to the AI model. The AI model (e.g., gradient boosting decision tree, random forest, Transformer-based large language model, etc.) integrally analyzes the input disease name, hospital data, and category information, and outputs optimal display algorithm selection instructions for each category (e.g., internal medicine: standard display; surgery: emphasize treatment records; psychiatry: emphasize reviews, etc.) and display item selection instructions. Examples of input to the AI model include disease name “gastroenteritis” category internal medicine, disease name “fracture” category surgery, disease name “depression” category psychiatry, hospital evaluation 4.8, treatment records 200 cases, etc. Examples of output from the AI model include “gastroenteritis” standard display, “fracture” emphasize treatment records, “depression” emphasize reviews, etc. The display unit automatically adjusts display items and layout for each disease name category on the user interface based on these AI outputs. For example, for surgical diseases, the display unit emphasizes surgical records and specialist information; for psychiatric diseases, the display unit switches to a display that emphasizes patient evaluations and reviews. These branches are controlled by the output scores of the AI model or rule-based branching logic. Furthermore, the display unit can link the category classification results to subsequent proposal units and estimation units to optimize the overall user experience. These processes differ from conventional uniform hospital information display or subjective judgment by human operators, as AI automatically determines the optimal display algorithm by combining multidimensional data analysis, category classification, and rule-based branching, thereby improving the appropriateness of information provision, user satisfaction, reducing the risk of misdiagnosis, and improving overall system usability, which are improvements in computer technology itself. Specific application fields include comprehensive medical support systems, triage support in emergency medical settings, telemedicine platforms, and chronic disease management systems.

The display unit can estimate the user's emotion and change the display order of the list of hospitals based on the estimated emotion of the user. For example, the display unit estimates the user's emotion. For example, the display unit may use facial recognition technology to estimate the user's emotion. The display unit may also use voice analysis technology to estimate the user's emotion. For example, the display unit analyzes the tone and speed of the user's voice to estimate emotion. Furthermore, the display unit can change the display order of the list of hospitals based on the user's emotion. For example, if the user is feeling anxious, the display unit displays the most reliable hospital first. If the user is relaxed, the display unit may adjust the order based on hospital ratings. If the user is in a hurry, the display unit displays hospitals that can provide rapid consultation first. By adjusting the display order of the list of hospitals according to the user's emotion, more appropriate information can be provided. Emotion estimation is realized, for example, by using an emotion engine or an emotion estimation function implemented by generative AI. Generative AI may be a text generation AI (e.g., LLM) or a multimodal generative AI, but is not limited to such examples. Some or all of the above-described processing in the display unit may be performed using AI or without using AI. For example, the display unit may input the user's emotion data to generative AI and have the generative AI perform emotion estimation. Specifically, the display unit simultaneously acquires facial images (e.g., 224×224 pixel RGB images), voice waveform data (e.g., 16 kHz sampling, 5-second audio clips), and text data at the time of input (e.g., short sentences or word sequences entered by the user) for emotion estimation, and inputs these as a multimodal input tensor to an AI model. For image data, the display unit applies a convolutional neural network (e.g., ResNet-based); for audio data, after spectrogram conversion, it applies a convolutional neural network or a recurrent neural network (e.g., LSTM); and for text data, it applies a Transformer-based large language model. A multimodal fusion layer (e.g., attention fusion layer) is provided to integrate these feature extraction results, and finally outputs emotion labels (e.g., stress, relaxation, anxiety, hurry, etc.) and probability distributions for each emotion (e.g., stress 0.70, relaxation 0.10, anxiety 0.15, hurry 0.05). Examples of input to the AI model include a stern facial image+text such as “I'm in a hurry”+a fast-spoken audio clip, or a calm facial image+text such as “I want to input slowly”+a calm voice audio clip. Examples of output from the AI model include emotion label “anxiety” probability 0.80, “relaxation” probability 0.10, “hurry” probability 0.10, or “relaxation” probability 0.85, “anxiety” probability 0.05, etc. The display unit automatically determines the display order of the list of hospitals based on these emotion estimation results. For example, if anxiety is high, hospitals with high reliability scores (e.g., hospitals with high treatment records, specialist qualifications, and patient evaluations) are displayed at the top; if the user is relaxed, hospitals are sorted by evaluation scores or reviews; if the user is in a hurry, hospitals available for immediate consultation or those closest to the current location are displayed at the top, and the display order is dynamically adjusted. These branches are controlled by the output probabilities of the AI model or rule-based branching logic. Furthermore, the display unit can link the emotion estimation results to subsequent proposal units and estimation units to optimize the overall user experience. These processes differ from conventional uniform hospital list displays or subjective judgments by human operators, as AI automatically determines the optimal display order by combining multimodal data analysis, probabilistic inference, and rule-based branching, thereby improving the appropriateness of information provision, user satisfaction, reducing the risk of misdiagnosis, and improving overall system usability, which are improvements in computer technology itself. Specific application fields include triage support in emergency medical settings, telemedicine platforms, chronic disease management systems, and corporate health management support services.

The display unit can determine the display priority based on the submission timing of the disease name when displaying the list of hospitals. For example, the display unit evaluates the submission timing of the disease name. For example, the display unit evaluates the submission timing based on the submission date and time or submission frequency of the disease name. The display unit can determine the display priority based on the submission timing of the disease name. For example, the display unit displays hospitals preferentially based on recently submitted disease names. The display unit may also adjust the display priority based on past disease names. The display unit determines the display priority according to the submission timing of the disease name. By determining the display priority according to the submission timing of the disease name, more rapid information provision becomes possible. Some or all of the above-described processing in the display unit may be performed using AI or without using AI. For example, the display unit may input the submission timing of the disease name to AI and have the AI determine the display priority. Specifically, the display unit stores a list of candidate disease names received from the estimation unit or analysis unit, along with the submission date and time for each disease name (e.g., Jun. 1, 2024, 10:23:00) and submission frequency (e.g., once per day, three times per week, etc.) as structured data with timestamps (e.g., 1×N dimensional vector+timestamp array) in a database. The display unit performs preprocessing such as time series normalization (e.g., standardization of submission intervals, encoding of time), aggregation of submission frequency, and weighting of submission timing (e.g., assigning higher weights to recent data), and shapes these as input tensors for display. The display unit uses an AI model such as a recurrent neural network (LSTM or GRU), time series Transformer, or a combination of multilayer perceptron and time series feature extraction module to simultaneously input the disease name candidate vector and submission timing information, and outputs a hospital list (e.g., hospital ID list with recommendation scores) and display priority scores for each hospital (e.g., Hospital A 0.95, Hospital B 0.60, etc.). Examples of input to the AI model include disease name “influenza” submission date Jun. 1, 2024, 10:23, submission frequency once per day; disease name “gastroenteritis” submission date May 25, 2024, 08:00, submission frequency once per week, etc. Examples of output from the AI model include Hospital A priority 0.95, Hospital B priority 0.60, Hospital C priority 0.30, etc. The display unit displays hospitals related to recent disease names at the top based on the output priority scores, and hospitals related to past disease names are displayed as supplementary information at the bottom. Furthermore, if the submission timing is continuous or the submission frequency is high, the display unit detects time-series changes in symptoms or signs of acute onset, and automatically adjusts the urgency and level of detail of the display results. These branches are controlled by threshold determination based on the output probabilities of the AI model or submission timing weighting parameters, and rule-based branching logic. Subsequent proposal units or estimation units automatically optimize treatment policy proposals or urgency display based on the prioritized hospital list received from the display unit. These processes differ from conventional uniform hospital display or subjective judgment by human operators, as AI automatically determines the optimal display priority by combining time-series data analysis, submission timing weighting, probabilistic inference, and rule-based branching, thereby speeding up information provision, enabling prompt response to emergency cases, improving user satisfaction, reducing the risk of misguidance, and improving overall system usability, which are improvements in computer technology itself. Specific application fields include triage support in emergency medical settings, acute exacerbation detection in chronic disease management systems, emergency alert issuance in remote health record systems, and early detection of health risks in corporate health management support services.

The display unit can adjust the display order based on the relevance of the disease name when displaying the list of hospitals. For example, the display unit evaluates the relevance of the disease name. For example, the display unit evaluates the relevance of the disease name based on common symptoms or onset patterns. The display unit can adjust the display order based on the relevance of the disease name. For example, the display unit displays the most relevant hospitals first based on the relevance of the disease name. The display unit may also adjust the display order according to the relevance of the disease name. The display unit analyzes the relevance of the disease name and optimizes the display order. By adjusting the display order based on the relevance of the disease name, more relevant information can be provided. Some or all of the above-described processing in the display unit may be performed using AI or without using AI. For example, the display unit may input the relevance of the disease name to AI and have the AI adjust the display order. Specifically, the display unit receives a list of candidate disease names (e.g., influenza, gastroenteritis, migraine, etc.) and symptom vectors or onset pattern data related to each disease name (e.g., co-occurrence of fever, headache, cough; co-occurrence of abdominal pain, diarrhea, etc.) from the estimation unit or analysis unit as 1×N dimensional numerical vectors or categorical data. The display unit performs preprocessing such as normalization (e.g., Z-score normalization), standardization, missing value imputation (e.g., median imputation), and outlier detection (e.g., IQR method or Z-score determination), and shapes these as input tensors for display. The display unit extracts features such as co-occurrence frequency of symptoms, time-series onset patterns, correlation coefficients with past diagnosis history, and disease name clustering results to evaluate the relevance between disease names. The AI model (e.g., graph neural network, Transformer-based large language model, or multilayer perceptron) inputs these features and calculates a relevance score matrix between disease names (e.g., relevance 0.0-1.0 for each disease name pair). Examples of input to the AI model include disease name “influenza”-“cold” relevance 0.85, disease name “gastroenteritis”-“migraine” relevance 0.10, co-occurrence patterns of fever, headache, cough, etc. Examples of output from the AI model include highly relevant disease name groups (e.g., influenza and cold), hospital lists with relevance scores (Hospital A 0.90, Hospital B 0.70, etc.), display priority (Hospital A 1st, Hospital B 2nd, etc.), etc. The display unit displays hospitals related to highly relevant disease name groups at the top, and hospitals corresponding to less relevant disease names at the bottom. Furthermore, when multiple disease names are estimated simultaneously, the display unit applies multitask learning models or multimodal fusion models to optimize the display order based on comprehensive relevance evaluation. These branches are controlled by the output scores of the AI model or rule-based branching logic. Subsequent proposal units or estimation units automatically optimize treatment policy proposals or hospital selection based on the relevance evaluation results received from the display unit. These processes differ from conventional simple disease name lists or subjective judgment by human operators, as AI automatically determines the optimal display order by combining feature extraction in high-dimensional vector space, relevance analysis between disease names, graph structure analysis, probabilistic inference, and rule-based branching, thereby improving the appropriateness of information provision, coverage of related diseases, reducing the risk of misguidance, improving user satisfaction, and improving overall system usability, which are improvements in computer technology itself. Specific application fields include comprehensive medical support systems, triage support in emergency medical settings, telemedicine platforms, chronic disease management systems, and corporate health management support services.

The analysis unit can estimate the user's emotion and change the analysis method of examination results based on the estimated emotion of the user. For example, the analysis unit estimates the user's emotion. For example, the analysis unit may use facial recognition technology to estimate the user's emotion. The analysis unit may also use voice analysis technology to estimate the user's emotion. For example, the analysis unit analyzes the tone and speed of the user's voice to estimate emotion. Furthermore, the analysis unit can change the analysis method of examination results based on the user's emotion. For example, if the user is feeling anxious, the analysis unit provides analysis results including detailed explanations. If the user is relaxed, the analysis unit may provide concise analysis results. If the user is in a hurry, the analysis unit provides analysis results quickly. By adjusting the analysis method of examination results according to the user's emotion, more appropriate analysis results can be provided. Emotion estimation is realized, for example, by using an emotion engine or an emotion estimation function implemented by generative AI. Generative AI may be a text generation AI (e.g., LLM) or a multimodal generative AI, but is not limited to such examples. Some or all of the above-described processing in the analysis unit may be performed using AI or without using AI. For example, the analysis unit may input the user's emotion data to generative AI and have the generative AI perform emotion estimation. Specifically, the analysis unit simultaneously acquires facial images (e.g., 224×224 pixel RGB images), voice waveform data (e.g., 16 kHz sampling, 5-second audio clips), and text data at the time of input (e.g., short sentences or word sequences entered by the user) for emotion estimation, and inputs these as a multimodal input tensor to an AI model. For image data, the analysis unit applies a convolutional neural network (e.g., ResNet-based); for audio data, after spectrogram conversion, it applies a convolutional neural network or a recurrent neural network (e.g., LSTM); and for text data, it applies a Transformer-based large language model. A multimodal fusion layer (e.g., attention fusion layer) is provided to integrate these feature extraction results, and finally outputs emotion labels (e.g., stress, relaxation, anxiety, hurry, etc.) and probability distributions for each emotion (e.g., stress 0.70, relaxation 0.10, anxiety 0.15, hurry 0.05). Examples of input to the AI model include a smiling facial image+text such as “I'm anxious about today's examination”+a calm voice audio clip, or a stern facial image+text such as “I'm in a hurry”+a fast-spoken audio clip. Examples of output from the AI model include emotion label “anxiety” probability 0.80, “relaxation” probability 0.10, “hurry” probability 0.10, or “relaxation” probability 0.85, “anxiety” probability 0.05, etc. The analysis unit automatically switches the branching logic of the examination result analysis algorithm based on these emotion estimation results. For example, if anxiety is high, the analysis unit utilizes the explanation generation function of a large language model to generate analysis results that describe in detail the meaning of examination values, the basis for abnormal values, the necessity of additional examinations, and treatment options. If the user is relaxed, the analysis unit displays only the main examination values and their normal/abnormal judgments concisely. If the user is in a hurry, the analysis unit omits part of the inference pipeline and immediately displays only the most important abnormal values or urgent findings, thereby speeding up the analysis process. These branches are controlled by threshold determination based on the output probabilities of the AI model or rule-based branching logic. Furthermore, the analysis unit can link the emotion estimation results to subsequent display units and proposal units to optimize the overall user experience. These processes differ from conventional uniform examination result analysis or subjective judgment by human operators, as the analysis unit automatically determines the optimal analysis method by combining multimodal data analysis, probabilistic inference, and rule-based branching, thereby improving analysis accuracy, user satisfaction, reducing the risk of misdiagnosis, and improving overall system usability, which are improvements in computer technology itself. Specific application fields include remote examination result notification systems, chronic disease management platforms, corporate health checkup support services, and rapid examination result feedback in emergency medical settings.

The analysis unit can adjust the level of detail of analysis based on the importance of the examination results when analyzing examination results. For example, the analysis unit evaluates the importance of the examination results. For example, the analysis unit evaluates the importance of the examination results based on the range of abnormal values and the importance of examination items. The analysis unit can adjust the level of detail of analysis based on the importance of the examination results. For example, the analysis unit provides detailed analysis results based on important examination results. The analysis unit may also provide concise analysis results based on minor examination results. The analysis unit adjusts the level of detail of analysis according to the importance of the examination results. By adjusting the level of detail of analysis according to the importance of the examination results, more accurate analysis results can be provided. Some or all of the above-described processing in the analysis unit may be performed using AI or without using AI. For example, the analysis unit may input the importance of the examination results to AI and have the AI adjust the level of detail of analysis. Specifically, the analysis unit receives examination result data submitted by the user (e.g., blood test values, imaging findings, vital signs, etc. as 1×N dimensional numerical vectors or categorical data). The analysis unit performs preprocessing such as normalization (e.g., Z-score normalization), standardization, missing value imputation (e.g., median imputation), and abnormal value detection (e.g., IQR method or Z-score determination), and shapes these as input tensors for analysis. The analysis unit sets weighting parameters for each examination item and calculates importance scores based on the degree of abnormal values, clinical significance, and relevance to disease risk. The AI model (e.g., multilayer perceptron, gradient boosting decision tree, Transformer-based large language model, etc.) combines the input examination result vectors and importance scores to perform inference, and outputs an analysis result list (e.g., abnormal value indication, disease risk estimation, treatment recommendation, etc.) and explanation detail level for each item (e.g., detailed explanation, concise explanation, key points only, etc.). Examples of input to the AI model include vector data such as blood glucose 250 mg/dL (high severity), white blood cell count 8000/μL (normal), CRP 0.2 mg/dL (minor), etc. Examples of output from the AI model include “blood glucose abnormality” with detailed explanation, “white blood cell count” with concise explanation, “CRP” with key points only, etc. The analysis unit generates analysis results that describe in detail the clinical significance of abnormal values, necessity of additional examinations, and treatment policy for examination items with high importance scores, and displays only the main abnormal values and concise explanations for items with low importance. These branches are controlled by threshold determination based on the output probabilities of the AI model or weighting parameters, and rule-based branching logic. Furthermore, the analysis unit can link the importance evaluation results to subsequent display units and proposal units to optimize the overall user experience. These processes differ from conventional uniform examination result analysis or subjective judgment by human operators, as AI automatically determines the optimal level of detail of analysis by combining feature extraction in high-dimensional vector space, weighting, probabilistic inference, and rule-based branching, thereby improving analysis accuracy, user satisfaction, reducing the risk of misdiagnosis, and improving overall system usability, which are improvements in computer technology itself. Specific application fields include emergency examination result feedback systems, chronic disease management platforms, corporate health checkup support services, and remote medical examination result notification systems.

The analysis unit can apply different analysis algorithms according to the category of the examination results when analyzing examination results. For example, the analysis unit classifies the category of the examination results. For example, the analysis unit classifies examination results into categories such as blood tests, imaging diagnosis, genetic tests, etc. The analysis unit can apply different analysis algorithms according to the category of the examination results. For example, the analysis unit applies a standard analysis algorithm to blood test results. The analysis unit may also apply a specialized analysis algorithm to MRI results. The analysis unit selects the optimal analysis algorithm according to the category of the examination results. By applying the optimal analysis algorithm according to the category of the examination results, more accurate analysis results can be provided. Some or all of the above-described processing in the analysis unit may be performed using AI or without using AI. For example, the analysis unit may input the category of the examination results to AI and have the AI apply the analysis algorithm. Specifically, the analysis unit receives examination result data submitted by the user (e.g., blood test values, MRI images, genetic sequence data, etc.) as 1×N dimensional numerical vectors, image tensors, or sequence data. The analysis unit performs preprocessing such as normalization, standardization, and category classification (e.g., blood test, imaging diagnosis, genetic test, vital signs, etc.), and selects different analysis algorithms for each category. For example, multilayer perceptron or gradient boosting decision tree for blood tests, convolutional neural networks (e.g., ResNet, EfficientNet) for imaging diagnosis, and recurrent neural networks or Transformer-based sequence analysis models for genetic tests. Examples of input to the AI model include blood test values (e.g., white blood cell count 8000/μL, blood glucose 110 mg/dL), MRI images (e.g., 256×256 pixel grayscale images), genetic sequences (e.g., ATCG . . . ), etc. Examples of output from the AI model include “suspected anemia,” “high risk of diabetes” for blood tests; “tumor region detected,” “abnormal shadow indicated” for imaging diagnosis; and “specific disease risk score” for genetic tests. The analysis unit automatically switches the branching logic of the analysis algorithm based on the category classification results and performs inference with models optimized for each category. Furthermore, when examination results from multiple categories are submitted simultaneously, the analysis unit may apply multimodal fusion models or multitask learning models to generate comprehensive analysis results. These processes differ from conventional uniform examination result analysis or subjective judgment by human operators, as AI automatically determines the optimal analysis method by combining feature extraction in high-dimensional vector space, category classification, algorithm branching, and multimodal inference, thereby improving analysis accuracy, reducing the risk of misdiagnosis, improving overall system flexibility, and improving usability, which are improvements in computer technology itself. Specific application fields include comprehensive examination result analysis systems, rapid diagnostic support in emergency medical settings, telemedicine platforms, and chronic disease management systems.

The analysis unit can estimate the user's emotion and change the display method of analysis results based on the estimated emotion of the user. For example, the analysis unit estimates the user's emotion. For example, the analysis unit may use facial recognition technology to estimate the user's emotion. The analysis unit may also use voice analysis technology to estimate the user's emotion. For example, the analysis unit analyzes the tone and speed of the user's voice to estimate emotion. Furthermore, the analysis unit can change the display method of analysis results based on the user's emotion. For example, if the user is feeling anxious, the analysis unit provides a simple and highly visible display method. If the user is relaxed, the analysis unit may provide a display method that includes detailed information. If the user is in a hurry, the analysis unit provides a display method that focuses on key points. By adjusting the display method of analysis results according to the user's emotion, more appropriate information can be provided. Emotion estimation is realized, for example, by using an emotion engine or an emotion estimation function implemented by generative AI. Generative AI may be a text generation AI (e.g., LLM) or a multimodal generative AI, but is not limited to such examples. Some or all of the above-described processing in the analysis unit may be performed using AI or without using AI. For example, the analysis unit may input the user's emotion data to generative AI and have the generative AI perform emotion estimation. Specifically, the analysis unit simultaneously acquires facial images (e.g., 224×224 pixel RGB images), voice waveform data (e.g., 16 kHz sampling, 5-second audio clips), and text data at the time of input (e.g., short sentences or word sequences entered by the user) for emotion estimation, and inputs these as a multimodal input tensor to an AI model. For image data, the analysis unit applies a convolutional neural network (e.g., ResNet-based); for audio data, after spectrogram conversion, it applies a convolutional neural network or a recurrent neural network (e.g., LSTM); and for text data, it applies a Transformer-based large language model. A multimodal fusion layer (e.g., attention fusion layer) is provided to integrate these feature extraction results, and finally outputs emotion labels (e.g., stress, relaxation, anxiety, hurry, etc.) and probability distributions for each emotion (e.g., stress 0.70, relaxation 0.10, anxiety 0.15, hurry 0.05). Examples of input to the AI model include a stern facial image+text such as “I'm in a hurry”+a fast-spoken audio clip, or a calm facial image+text such as “I want to view slowly”+a calm voice audio clip. Examples of output from the AI model include emotion label “anxiety” probability 0.80, “relaxation” probability 0.10, “hurry” probability 0.10, or “relaxation” probability 0.85, “anxiety” probability 0.05, etc. The analysis unit automatically switches the display method of analysis results based on these emotion estimation results. For example, if anxiety is high, only the main abnormal values and treatment policy are displayed in large, simple text, and detailed information is collapsed. If the user is relaxed, trend graphs of examination values, detailed explanations, and reference links are expanded and displayed. If the user is in a hurry, only urgent abnormal values and key points are summarized and displayed, and the UI layout and amount of information are dynamically adjusted. These branches are controlled by the output probabilities of the AI model or rule-based branching logic. Furthermore, the analysis unit can link the emotion estimation results to subsequent display units and proposal units to optimize the overall user experience. These processes differ from conventional uniform examination result display or subjective judgment by human operators, as AI automatically determines the optimal display method by combining multimodal data analysis, probabilistic inference, and rule-based branching, thereby improving the appropriateness of information provision, user satisfaction, completion rate of input, and overall system usability, which are improvements in computer technology itself. Specific application fields include remote examination result notification systems, chronic disease management platforms, corporate health checkup support services, and rapid examination result feedback in emergency medical settings.

The analysis unit can determine the analysis priority based on the submission timing of the examination results when analyzing examination results. For example, the analysis unit evaluates the submission timing of the examination results. For example, the analysis unit evaluates the submission timing based on the submission date and time or submission frequency of the examination results. The analysis unit can determine the analysis priority based on the submission timing of the examination results. For example, the analysis unit prioritizes analysis based on recently submitted examination results. The analysis unit may also adjust the analysis priority based on past examination results. The analysis unit determines the analysis priority according to the submission timing of the examination results. By determining the analysis priority according to the submission timing of the examination results, more rapid analysis results can be provided. Some or all of the above-described processing in the analysis unit may be performed using AI or without using AI. For example, the analysis unit may input the submission timing of the examination results to AI and have the AI determine the analysis priority. Specifically, the analysis unit stores examination result data submitted by the user, along with the submission date and time (e.g., Jun. 1, 2024, 10:23:00) and submission frequency (e.g., once per day, three times per week, etc.) as structured data with timestamps (e.g., 1×N dimensional vector+timestamp array) in a database. The analysis unit performs preprocessing such as time series normalization (e.g., standardization of submission intervals, encoding of time), aggregation of submission frequency, and weighting of submission timing (e.g., assigning higher weights to recent data), and shapes these as input tensors for analysis. The analysis unit uses an AI model such as a recurrent neural network (LSTM or GRU), time series Transformer, or a combination of multilayer perceptron and time series feature extraction module to simultaneously input the examination result vector and submission timing information, and outputs an analysis result list (e.g., abnormal value indication, disease risk estimation, treatment recommendation, etc.) and priority scores for each analysis item (e.g., blood glucose 0.90, white blood cell count 0.60, etc.). Examples of input to the AI model include blood glucose 250 mg/dL, submission date Jun. 1, 2024, 10:23, submission frequency once per day; or CRP 0.2 mg/dL, submission date May 25, 2024, 08:00, submission frequency once per week, etc. Examples of output from the AI model include “blood glucose abnormality” priority 0.95, “white blood cell count” priority 0.60, “CRP” priority 0.30, etc. The analysis unit prioritizes analysis of recent examination results based on the output priority scores, and past examination results are weighted as supplementary reference information within the analysis algorithm. Furthermore, if the submission timing is continuous or the submission frequency is high, the analysis unit detects time-series changes in examination values or signs of acute deterioration, and automatically adjusts the urgency and level of detail of the analysis results. These branches are controlled by threshold determination based on the output probabilities of the AI model or submission timing weighting parameters, and rule-based branching logic. Subsequent display units or proposal units automatically optimize treatment policy proposals or urgency display based on the prioritized analysis result list received from the analysis unit. These processes differ from conventional uniform examination result analysis or subjective judgment by human operators, as AI automatically determines the optimal analysis priority by combining time-series data analysis, submission timing weighting, probabilistic inference, and rule-based branching, thereby improving analysis accuracy, enabling rapid response to emergency cases, improving user satisfaction, reducing the risk of misdiagnosis, and improving overall system usability, which are improvements in computer technology itself. Specific application fields include emergency examination result feedback systems, chronic disease management platforms, remote medical examination result notification systems, and corporate health checkup support services.

The analysis unit can adjust the analysis order based on the relevance of the examination results when analyzing examination results. For example, the analysis unit evaluates the relevance of the examination results. For example, the analysis unit evaluates the relevance of the examination results based on common abnormal values or the relevance of examination items. The analysis unit can adjust the analysis order based on the relevance of the examination results. For example, the analysis unit analyzes the most relevant results first based on the relevance of the examination results. The analysis unit may also adjust the analysis order according to the relevance of the examination results. The analysis unit analyzes the relevance of the examination results and optimizes the analysis order. By adjusting the analysis order based on the relevance of the examination results, more relevant information can be provided. Some or all of the above-described processing in the analysis unit may be performed using AI or without using AI. For example, the analysis unit may input the relevance of the examination results to AI and have the AI adjust the analysis order. Specifically, the analysis unit receives examination result data submitted by the user (e.g., blood test values, imaging findings, vital signs, etc. as 1×N dimensional numerical vectors or categorical data). The analysis unit performs preprocessing such as normalization (e.g., Z-score normalization), standardization, missing value imputation (e.g., median imputation), and abnormal value detection (e.g., IQR method or Z-score determination), and shapes these as input tensors for analysis. The analysis unit extracts features such as co-occurrence frequency of abnormal values, time-series patterns of examination values, correlation coefficients with past diagnosis history, and clustering results of examination items to evaluate the relevance between examination items. The AI model (e.g., graph neural network, Transformer-based large language model, or multilayer perceptron) inputs these features and calculates a relevance score matrix between examination items (e.g., relevance 0.0-1.0 for each item pair). Examples of input to the AI model include co-occurrence patterns of blood glucose, HbA1c, and insulin values; time-series variation of white blood cell count, CRP, and body temperature; examination history vectors for the past 7 days, etc. Examples of output from the AI model include relevance scores between examination items (e.g., blood glucose-HbA1 c 0.90, white blood cell count-CRP 0.85), highly relevant examination groups (e.g., inflammation marker group), analysis result list (e.g., high risk of diabetes, suspected infection, etc.), and display priority for each analysis item (e.g., diabetes 1st, infection 2nd, etc.). The analysis unit automatically determines the display order of analysis results based on highly relevant examination groups. For example, if the relevance between blood glucose, HbA1c, and insulin values is high, diabetes-related analysis results are displayed at the top; if the relevance between white blood cell count, CRP, and body temperature is high, infection-related analysis results are displayed at the top. These branches are controlled by the output scores of the AI model or rule-based branching logic. Furthermore, the analysis unit can link the relevance evaluation results to subsequent display units and proposal units to optimize treatment policy proposals and hospital selection. These processes differ from conventional simple examination item lists or subjective judgment by human operators, as AI automatically determines the optimal analysis order by combining feature extraction in high-dimensional vector space, relevance analysis between examination items, graph structure analysis, probabilistic inference, and rule-based branching, thereby improving analysis accuracy, coverage of related diseases, reducing the risk of misdiagnosis, improving user satisfaction, and improving overall system usability, which are improvements in computer technology itself. Specific application fields include comprehensive examination result analysis systems, rapid diagnostic support in emergency medical settings, telemedicine platforms, and chronic disease management systems.

The proposal unit can estimate the user's emotion and change the hospital proposal method based on the estimated emotion of the user. For example, the proposal unit estimates the user's emotion. For example, the proposal unit may use facial recognition technology to estimate the user's emotion. The proposal unit may also use voice analysis technology to estimate the user's emotion. For example, the proposal unit analyzes the tone and speed of the user's voice to estimate emotion. Furthermore, the proposal unit can change the hospital proposal method based on the user's emotion. For example, if the user is feeling anxious, the proposal unit provides hospital proposals including detailed explanations. If the user is relaxed, the proposal unit may provide concise hospital proposals. If the user is in a hurry, the proposal unit provides hospital proposals quickly. By adjusting the hospital proposal method according to the user's emotion, more appropriate proposals can be made. Emotion estimation is realized, for example, by using an emotion engine or an emotion estimation function implemented by generative AI. Generative AI may be a text generation AI (e.g., LLM) or a multimodal generative AI, but is not limited to such examples. Some or all of the above-described processing in the proposal unit may be performed using AI or without using AI. For example, the proposal unit may input the user's emotion data to generative AI and have the generative AI perform emotion estimation.

The proposal unit can adjust the level of detail of proposals based on the importance of the examination results when proposing hospitals. For example, the proposal unit evaluates the importance of the examination results. For example, the proposal unit evaluates the importance of the examination results based on the range of abnormal values and the importance of examination items. The proposal unit can adjust the level of detail of proposals based on the importance of the examination results. For example, the proposal unit provides detailed hospital proposals based on important examination results. The proposal unit may also provide concise hospital proposals based on minor examination results. The proposal unit adjusts the level of detail of proposals according to the importance of the examination results. By adjusting the level of detail of proposals according to the importance of the examination results, more accurate proposals can be made. Some or all of the above-described processing in the proposal unit may be performed using AI or without using AI. For example, the proposal unit may input the importance of the examination results to AI and have the AI adjust the level of detail of proposals.

The proposal unit can apply different proposal algorithms according to the category of the examination results when proposing hospitals. For example, the proposal unit classifies the category of the examination results. For example, the proposal unit classifies examination results into categories such as blood tests, imaging diagnosis, genetic tests, etc. The proposal unit can apply different proposal algorithms according to the category of the examination results. For example, the proposal unit applies a standard proposal algorithm to blood test results. The proposal unit may also apply a specialized proposal algorithm to MRI results. The proposal unit selects the optimal proposal algorithm according to the category of the examination results. By applying the optimal proposal algorithm according to the category of the examination results, more accurate proposals can be made. Some or all of the above-described processing in the proposal unit may be performed using AI or without using AI. For example, the proposal unit may input the category of the examination results to AI and have the AI apply the proposal algorithm.

The proposal unit can estimate the user's emotion and change the order of hospital proposals based on the estimated emotion of the user. For example, the proposal unit estimates the user's emotion. For example, the proposal unit may use facial recognition technology to estimate the user's emotion. The proposal unit may also use voice analysis technology to estimate the user's emotion. For example, the proposal unit analyzes the tone and speed of the user's voice to estimate emotion. Furthermore, the proposal unit can change the order of hospital proposals based on the user's emotion. For example, if the user is feeling anxious, the proposal unit proposes the most reliable hospital first. If the user is relaxed, the proposal unit may adjust the order based on hospital ratings. If the user is in a hurry, the proposal unit proposes hospitals that can provide rapid consultation first. By adjusting the order of hospital proposals according to the user's emotion, more appropriate proposals can be made. Emotion estimation is realized, for example, by using an emotion engine or an emotion estimation function implemented by generative AI. Generative AI may be a text generation AI (e.g., LLM) or a multimodal generative AI, but is not limited to such examples. Some or all of the above-described processing in the proposal unit may be performed using AI or without using AI. For example, the proposal unit may input the user's emotion data to generative AI and have the generative AI perform emotion estimation.

The proposal unit can determine the proposal priority based on the submission timing of the examination results when proposing hospitals. For example, the proposal unit evaluates the submission timing of the examination results. For example, the proposal unit evaluates the submission timing based on the submission date and time or submission frequency of the examination results. The proposal unit can determine the proposal priority based on the submission timing of the examination results. For example, the proposal unit proposes hospitals preferentially based on recently submitted examination results. The proposal unit may also adjust the proposal priority based on past examination results. The proposal unit determines the proposal priority according to the submission timing of the examination results. By determining the proposal priority according to the submission timing of the examination results, more rapid proposals can be made. Some or all of the above-described processing in the proposal unit may be performed using AI or without using AI. For example, the proposal unit may input the submission timing of the examination results to AI and have the AI determine the proposal priority.

The proposal unit can adjust the order of proposals based on the relevance of the examination results when proposing hospitals. For example, the proposal unit evaluates the relevance of the examination results. For example, the proposal unit evaluates the relevance of the examination results based on common abnormal values or the relevance of examination items. The proposal unit can adjust the order of proposals based on the relevance of the examination results. For example, the proposal unit proposes the most relevant hospitals first based on the relevance of the examination results. The proposal unit may also adjust the order of proposals according to the relevance of the examination results. The proposal unit analyzes the relevance of the examination results and optimizes the order of proposals. By adjusting the order of proposals based on the relevance of the examination results, more relevant proposals can be made. Some or all of the above-described processing in the proposal unit may be performed using AI or without using AI. For example, the proposal unit may input the relevance of the examination results to AI and have the AI adjust the order of proposals.

The system according to the embodiment is not limited to the examples described above, and various modifications are possible, for example, as follows.

The receiving unit can allow the user to input records of lifestyle habits and meals when receiving the user's physical condition information. For example, the receiving unit provides an interface for the user to input daily meal contents and amount of exercise, and stores this information in a database. The receiving unit may also periodically record information related to the user's lifestyle habits and utilize it for long-term health management. By integrally managing the user's physical condition information and lifestyle habit information, more accurate understanding of the health status becomes possible.

The analysis unit can take into account the user's genetic information when analyzing the user's physical condition information. For example, the analysis unit may store the user's genetic test results in a database and perform analysis by combining the physical condition information with the genetic information. In addition, the analysis unit can evaluate the risk of specific diseases based on genetic information and propose preventive measures. This enables more accurate analysis that considers the user's genetic background.

The proposal unit can provide advice for health management based on the results of analyzing the user's physical condition information. For example, the proposal unit may propose an appropriate exercise plan or diet plan based on the user's physical condition information. Furthermore, the proposal unit can provide advice for stress management and sleep improvement based on the user's physical condition information. This allows the user to receive specific advice for maintaining health in daily life.

The estimation unit can take into account the effects of seasons and weather when analyzing the user's physical condition information. For example, the estimation unit may store trends in disease occurrence by season and changes in physical condition due to weather changes in a database and reflect this information in the analysis. In addition, the estimation unit can provide health management advice according to the season and weather. This enables more accurate disease name estimation that considers the effects of seasons and weather.

The display unit can provide educational content related to health based on the results of analyzing the user's physical condition information. For example, the display unit may display information about specific diseases and preventive measures based on the user's physical condition information. Furthermore, the display unit can provide videos and articles related to health, offering the user opportunities to learn about health. This allows the user to deepen their knowledge about their own health.

The receiving unit can estimate the user's emotion and customize the input interface for physical condition information based on the estimated emotion of the user. For example, if the user is feeling stressed, the receiving unit may provide a simple and intuitive interface to reduce the effort required for input. In addition, if the user is relaxed, the receiving unit may provide detailed input options to allow the user to input more information. By adjusting the input interface according to the user's emotion, more appropriate input can be achieved.

The estimation unit can estimate the user's emotion and change the method of displaying disease name estimation results based on the estimated emotion of the user. For example, if the user is feeling anxious, the estimation unit may provide disease name estimation results with detailed explanations to provide reassurance. In addition, if the user is relaxed, the estimation unit may provide concise disease name estimation results. By adjusting the method of displaying disease name estimation results according to the user's emotion, more appropriate information provision can be achieved.

The analysis unit can estimate the user's emotion and change the method of analyzing examination results based on the estimated emotion of the user. For example, if the user is feeling anxious, the analysis unit may provide analysis results with detailed explanations to provide reassurance. In addition, if the user is relaxed, the analysis unit may provide concise analysis results. By adjusting the method of analyzing examination results according to the user's emotion, more appropriate analysis results can be provided.

The proposal unit can estimate the user's emotion and change the method of proposing hospitals based on the estimated emotion of the user. For example, if the user is feeling anxious, the proposal unit may propose hospitals with detailed explanations. In addition, if the user is relaxed, the proposal unit may propose hospitals concisely. By adjusting the method of proposing hospitals according to the user's emotion, more appropriate proposals can be made.

The display unit can estimate the user's emotion and change the method of displaying the list of hospitals based on the estimated emotion of the user. For example, if the user is feeling anxious, the display unit may provide a simple and highly visible display method. In addition, if the user is relaxed, the display unit may provide a display method including detailed information. By adjusting the method of displaying the list of hospitals according to the user's emotion, more appropriate information can be provided.

The following is a brief description of the processing flow of Example of the Embodiment.

Step 1: The receiving unit receives the user's physical condition information. The user's physical condition information includes body temperature, blood pressure, heart rate, details of symptoms, and the like. The receiving unit can record information such as body temperature, nausea, headache, etc. entered by the user and store it in a database.

Step 2: The estimation unit analyzes the physical condition information received by the receiving unit and estimates a disease name. The estimation unit uses AI to analyze the physical condition information and estimates a disease name by analyzing physical conditions such as body temperature, nausea, headache, and the location and degree of pain.

Step 3: The display unit displays a hospital to be visited based on the disease name estimated by the estimation unit. The display unit selects a hospital by considering the specialty, distance, and evaluation of the hospital based on the estimated disease name and displays it to the user.

Step 4: The analysis unit analyzes examination results after the visit. The analysis unit analyzes results of blood tests, MRI, CRI, X-ray, and ultrasound examinations and uses AI to estimate a disease name and treatment policy.

Step 5: The proposal unit proposes a hospital to be referred to based on the results analyzed by the analysis unit. The proposal unit identifies a hospital based on specialist data and proposes a hospital to be visited by the user.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned receiving unit, estimation unit, display unit, analysis unit, and proposal unit is implemented by at least one of, for example, the smart device 14 and the data processing apparatus 12. For example, the receiving unit is implemented by a receiving device 38 of the smart device 14 and records information such as body temperature, nausea, and headache input by the user. The estimation unit is implemented, for example, by a specific processing unit 290 of the data processing apparatus 12 and analyzes physical condition information using AI to estimate a disease name. The display unit is implemented, for example, by an output device 40 of the smart device 14 and displays a list of hospitals to be visited based on the estimated disease name. The analysis unit is implemented, for example, by the specific processing unit 290 of the data processing apparatus 12 and analyzes results of blood tests, MRI, CRI, X-ray, and ultrasound examinations. The proposal unit is implemented, for example, by the specific processing unit 290 of the data processing apparatus 12 and proposes a hospital to be referred to based on the analyzed results. The correspondence between each unit and the devices or control units is not limited to the examples described above and various modifications are possible.

### Second Embodiment

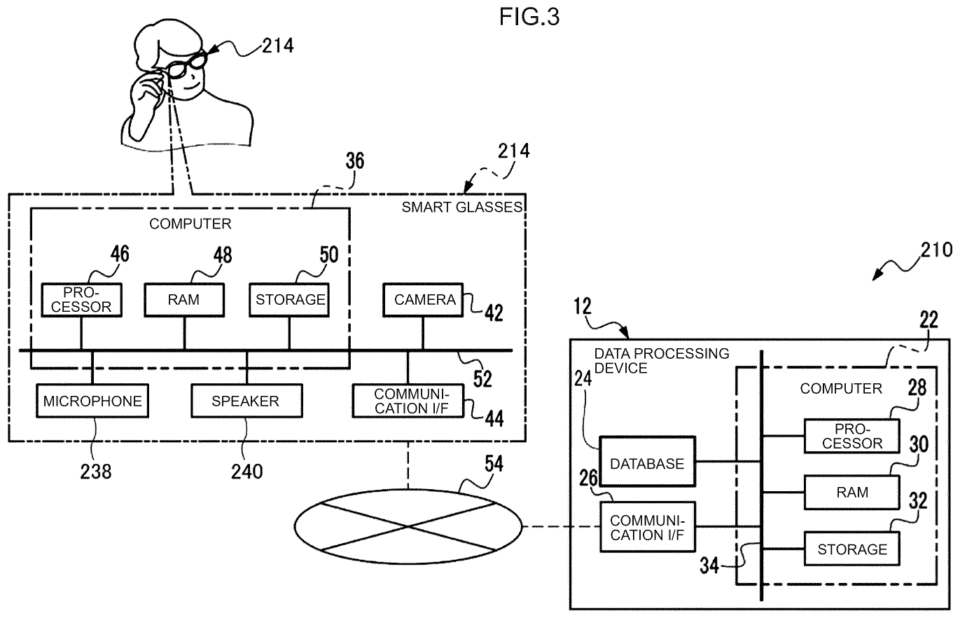

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

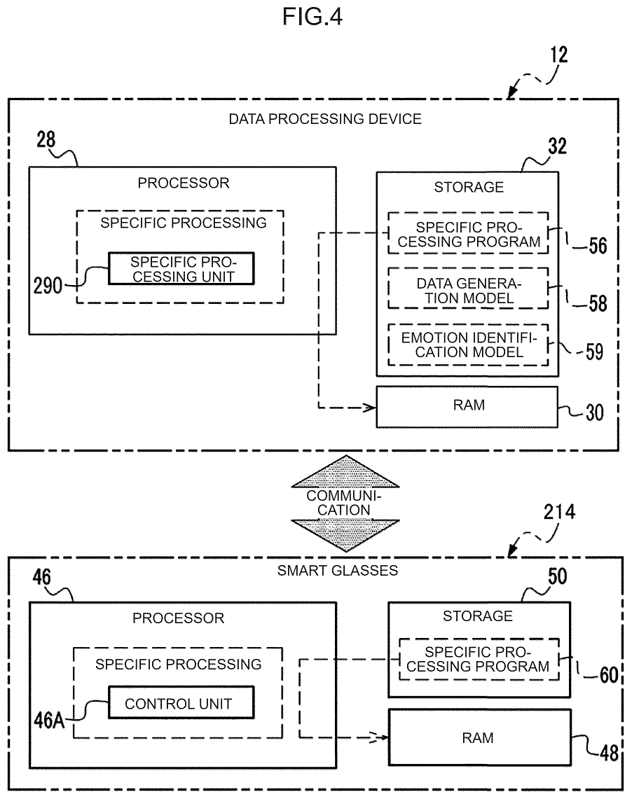

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned receiving unit, estimation unit, display unit, analysis unit, and proposal unit is implemented by at least one of, for example, the smart glasses 214 and the data processing apparatus 12. For example, the receiving unit is implemented by a microphone 238 of the smart glasses 214 and records information such as body temperature, nausea, and headache input by the user. The estimation unit is implemented, for example, by a specific processing unit 290 of the data processing apparatus 12 and analyzes physical condition information using AI to estimate a disease name. The display unit is implemented, for example, by a speaker 240 of the smart glasses 214 and displays a list of hospitals to be visited based on the estimated disease name. The analysis unit is implemented, for example, by the specific processing unit 290 of the data processing apparatus 12 and analyzes results of blood tests, MRI, CRI, X-ray, and ultrasound examinations. The proposal unit is implemented, for example, by the specific processing unit 290 of the data processing apparatus 12 and proposes a hospital to be referred to based on the analyzed results. The correspondence between each unit and the devices or control units is not limited to the examples described above and various modifications are possible.

### Third Embodiment

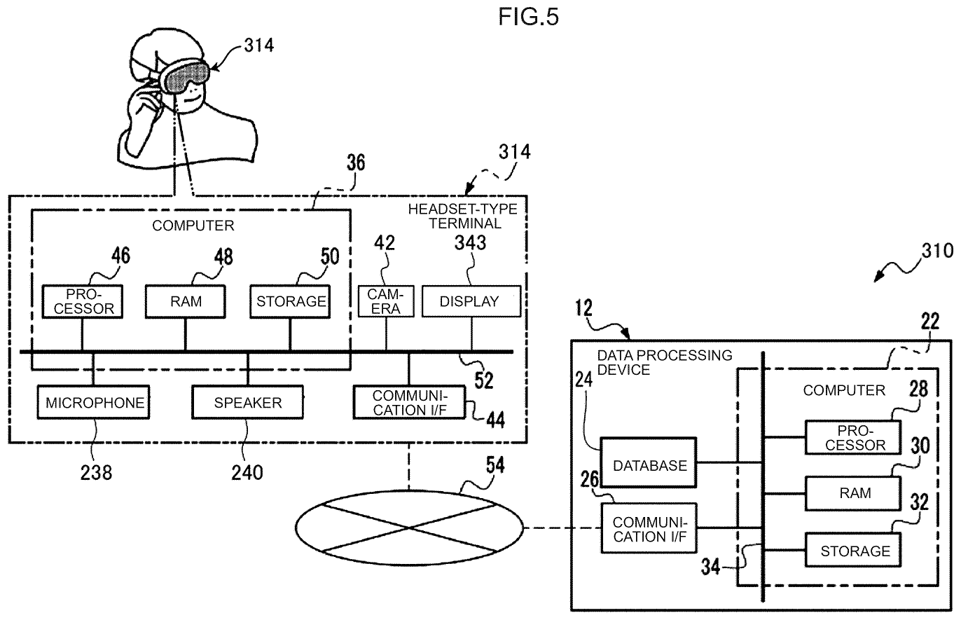

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

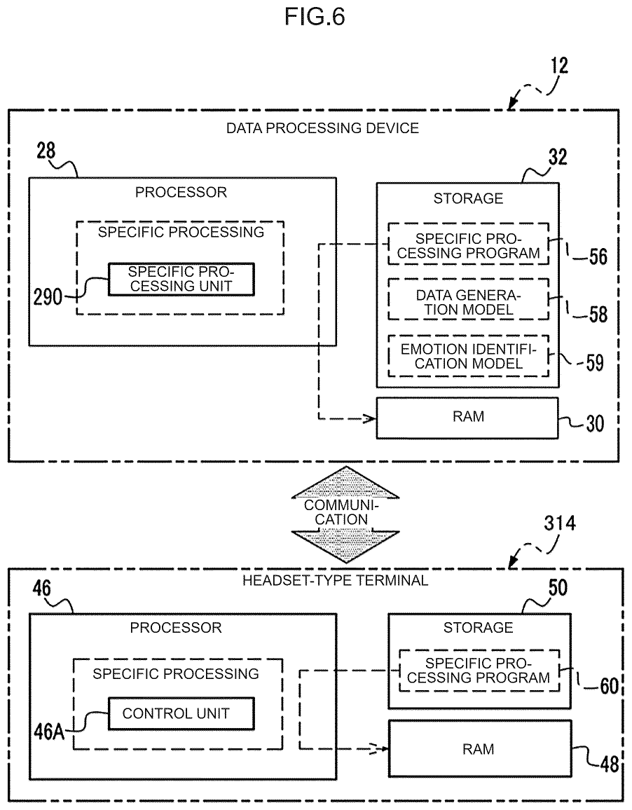

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned receiving unit, estimation unit, display unit, analysis unit, and proposal unit is implemented by at least one of, for example, the headset-type terminal 314 and the data processing apparatus 12. For example, the receiving unit is implemented by a microphone 238 of the headset-type terminal 314 and records information such as body temperature, nausea, and headache input by the user. The estimation unit is implemented, for example, by a specific processing unit 290 of the data processing apparatus 12 and analyzes physical condition information using AI to estimate a disease name. The display unit is implemented, for example, by a display 343 of the headset-type terminal 314 and displays a list of hospitals to be visited based on the estimated disease name. The analysis unit is implemented, for example, by the specific processing unit 290 of the data processing apparatus 12 and analyzes results of blood tests, MRI, CRI, X-ray, and ultrasound examinations. The proposal unit is implemented, for example, by the specific processing unit 290 of the data processing apparatus 12 and proposes a hospital to be referred to based on the analyzed results. The correspondence between each unit and the devices or control units is not limited to the examples described above and various modifications are possible.

### Fourth Embodiment

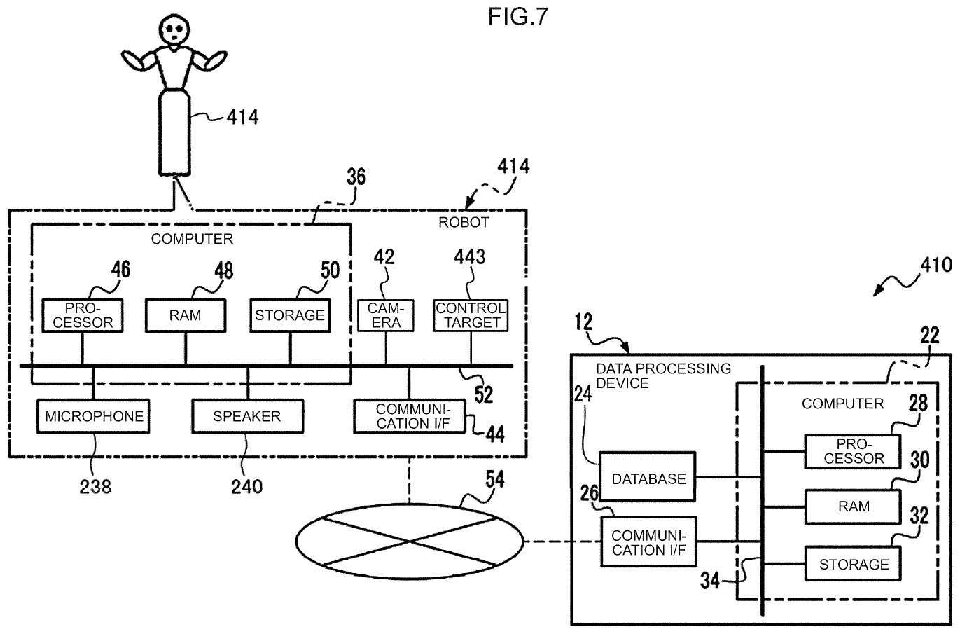

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

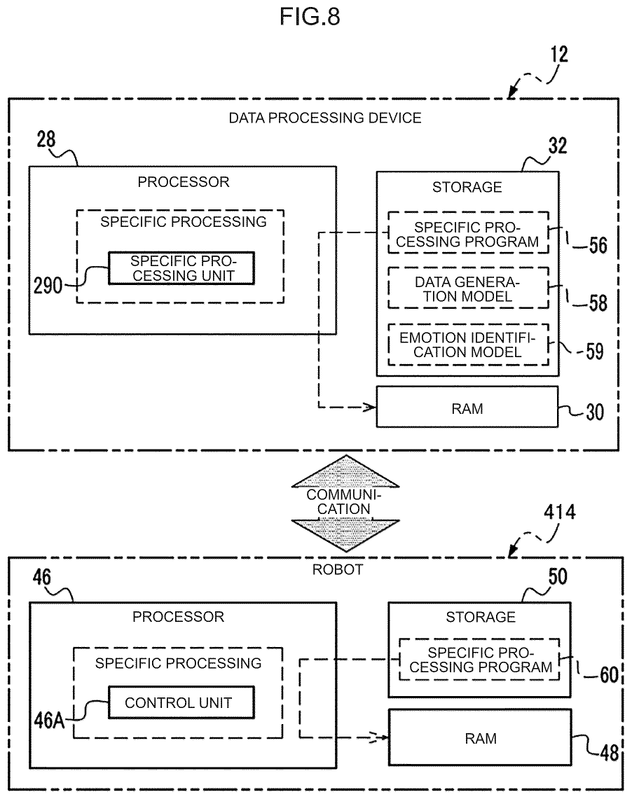

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned receiving unit, estimation unit, display unit, analysis unit, and proposal unit is implemented by at least one of, for example, the robot 414 and the data processing apparatus 12. For example, the receiving unit is implemented by a microphone 238 of the robot 414 and records information such as body temperature, nausea, and headache input by the user. The estimation unit is implemented, for example, by a specific processing unit 290 of the data processing apparatus 12 and analyzes physical condition information using AI to estimate a disease name. The display unit is implemented, for example, by a speaker 240 of the robot 414 and displays a list of hospitals to be visited based on the estimated disease name. The analysis unit is implemented, for example, by the specific processing unit 290 of the data processing apparatus 12 and analyzes results of blood tests, MRI, CRI, X-ray, and ultrasound examinations. The proposal unit is implemented, for example, by the specific processing unit 290 of the data processing apparatus 12 and proposes a hospital to be referred to based on the analyzed results. The correspondence between each unit and the devices or control units is not limited to the examples described above and various modifications are possible.

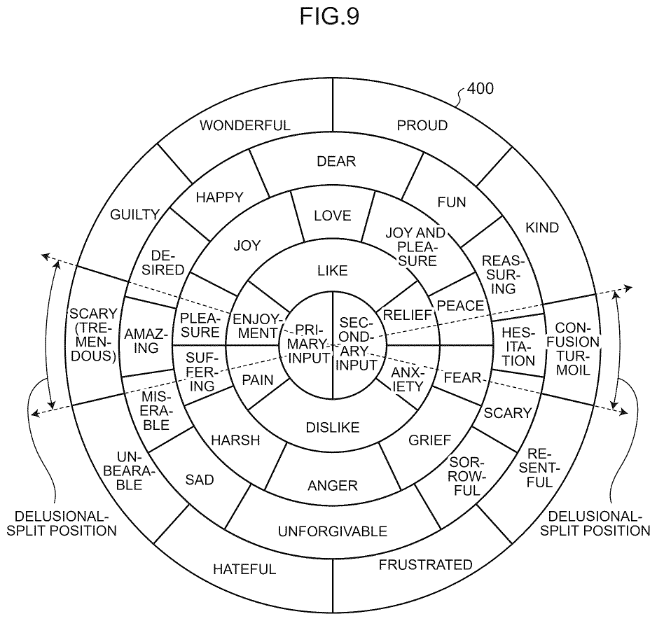

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

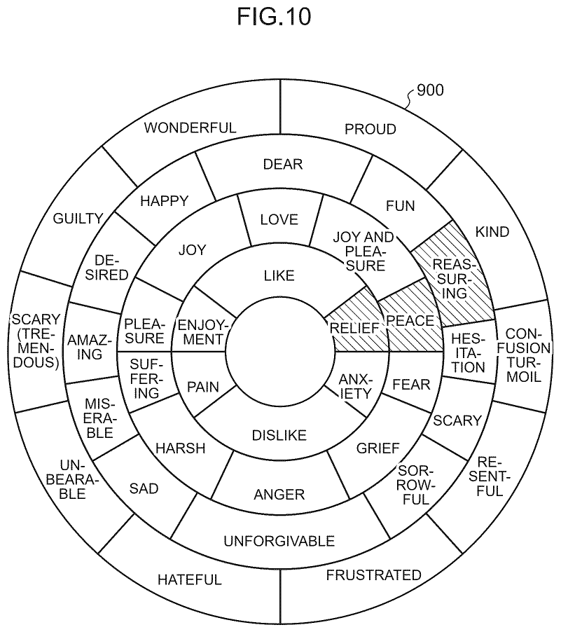

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

(Supplementary Note 1) A system comprising: a receiving unit configured to receive physical condition information of a user; an estimation unit configured to analyze the physical condition information received by the receiving unit and estimate a disease name; a display unit configured to display a hospital to be visited based on the disease name estimated by the estimation unit; an analysis unit configured to analyze examination results after the visit; and a proposal unit configured to propose a hospital to be referred to based on the results analyzed by the analysis unit.

(Supplementary Note 2) The system according to Supplementary Note 1, wherein the receiving unit is configured to record the physical condition information of the user.

(Supplementary Note 3) The system according to Supplementary Note 1, wherein the analysis unit is configured to analyze results of blood tests, MRI, CRI, X-ray, and ultrasound examinations.

(Supplementary Note 4) The system according to Supplementary Note 1, wherein the proposal unit is configured to identify a hospital based on specialist data.

(Supplementary Note 5) The system according to Supplementary Note 1, wherein the estimation unit is configured to estimate a disease name by analyzing physical conditions such as body temperature, nausea, headache, and the location and degree of pain.

(Supplementary Note 6) The system according to Supplementary Note 1, wherein the display unit is configured to display a list of hospitals to be visited based on the estimated disease name.

(Supplementary Note 7) The system according to Supplementary Note 1, wherein the receiving unit is configured to estimate the user's emotion and change the input method for physical condition information based on the estimated emotion of the user.

(Supplementary Note 8) The system according to Supplementary Note 1, wherein the receiving unit is configured to analyze the user's past physical condition information and provide an appropriate input format.

(Supplementary Note 9) The system according to Supplementary Note 1, wherein the receiving unit is configured to perform filtering based on the user's lifestyle habits and medical history when inputting physical condition information.

(Supplementary Note 10) The system according to Supplementary Note 1, wherein the receiving unit is configured to estimate the user's emotion and determine the order of physical condition information to be input based on the estimated emotion of the user.

(Supplementary Note 11) The system according to Supplementary Note 1, wherein the receiving unit is configured to consider the user's geographic location information when inputting physical condition information and prioritize the input of highly relevant information.

(Supplementary Note 12) The system according to Supplementary Note 1, wherein the receiving unit is configured to analyze the user's social media activity when inputting physical condition information and input relevant information.

(Supplementary Note 13) The system according to Supplementary Note 1, wherein the estimation unit is configured to estimate the user's emotion and change the disease name estimation method based on the estimated emotion of the user.

(Supplementary Note 14) The system according to Supplementary Note 1, wherein the estimation unit is configured to adjust the level of detail of estimation based on the importance of the physical condition information when estimating a disease name.

(Supplementary Note 15) The system according to Supplementary Note 1, wherein the estimation unit is configured to apply different estimation algorithms according to the category of physical condition information when estimating a disease name.

(Supplementary Note 16) The system according to Supplementary Note 1, wherein the estimation unit is configured to estimate the user's emotion and change the order of displaying disease name estimation results based on the estimated emotion of the user.

(Supplementary Note 17) The system according to Supplementary Note 1, wherein the estimation unit is configured to determine the priority of estimation based on the submission timing of the physical condition information when estimating a disease name.

(Supplementary Note 18) The system according to Supplementary Note 1, wherein the estimation unit is configured to adjust the order of estimation based on the relevance of the physical condition information when estimating a disease name.

(Supplementary Note 19) The system according to Supplementary Note 1, wherein the display unit is configured to estimate the user's emotion and change the display method of the list of hospitals based on the estimated emotion of the user.

(Supplementary Note 20) The system according to Supplementary Note 1, wherein the display unit is configured to adjust the level of detail of display based on the importance of the disease name when displaying the list of hospitals.

(Supplementary Note 21) The system according to Supplementary Note 1, wherein the display unit is configured to apply different display algorithms according to the category of the disease name when displaying the list of hospitals.

(Supplementary Note 22) The system according to Supplementary Note 1, wherein the display unit is configured to estimate the user's emotion and change the display order of the list of hospitals based on the estimated emotion of the user.

(Supplementary Note 23) The system according to Supplementary Note 1, wherein the display unit is configured to determine the priority of display based on the submission timing of the disease name when displaying the list of hospitals.

(Supplementary Note 24) The system according to Supplementary Note 1, wherein the display unit is configured to adjust the display order based on the relevance of the disease name when displaying the list of hospitals.

(Supplementary Note 25) The system according to Supplementary Note 1, wherein the analysis unit is configured to estimate the user's emotion and change the analysis method of examination results based on the estimated emotion of the user.

(Supplementary Note 26) The system according to Supplementary Note 1, wherein the analysis unit is configured to adjust the level of detail of analysis based on the importance of the examination results when analyzing examination results.

(Supplementary Note 27) The system according to Supplementary Note 1, wherein the analysis unit is configured to apply different analysis algorithms according to the category of the examination results when analyzing examination results.

(Supplementary Note 28) The system according to Supplementary Note 1, wherein the analysis unit is configured to estimate the user's emotion and change the display method of analysis results based on the estimated emotion of the user.

(Supplementary Note 29) The system according to Supplementary Note 1, wherein the analysis unit is configured to determine the priority of analysis based on the submission timing of the examination results when analyzing examination results.

(Supplementary Note 30) The system according to Supplementary Note 1, wherein the analysis unit is configured to adjust the order of analysis based on the relevance of the examination results when analyzing examination results.

(Supplementary Note 31) The system according to Supplementary Note 1, wherein the proposal unit is configured to estimate the user's emotion and change the hospital proposal method based on the estimated emotion of the user.

(Supplementary Note 32) The system according to Supplementary Note 1, wherein the proposal unit is configured to adjust the level of detail of proposal based on the importance of the examination results when proposing a hospital.

(Supplementary Note 33) The system according to Supplementary Note 1, wherein the proposal unit is configured to apply different proposal algorithms according to the category of the examination results when proposing a hospital.

(Supplementary Note 34) The system according to Supplementary Note 1, wherein the proposal unit is configured to estimate the user's emotion and change the order of hospital proposals based on the estimated emotion of the user.

(Supplementary Note 35) The system according to Supplementary Note 1, wherein the proposal unit is configured to determine the priority of proposal based on the submission timing of the examination results when proposing a hospital.

(Supplementary Note 36) The system according to Supplementary Note 1, wherein the proposal unit is configured to adjust the order of proposal based on the relevance of the examination results when proposing a hospital.

What is claimed is:

1. A system comprising:
circuitry configured to:
receive, from a client terminal via a packet-switched network, an input feature vector encoding a plurality of numerical measurements;
generate, by inputting the input feature vector as a multidimensional tensor into a first neural network comprising a Transformer-based architecture with attention layers, a set of classification labels each associated with a confidence score;
retrieve, from a database, a plurality of reference records corresponding to the set of classification labels, and generate a ranked list of the reference records by applying a scoring function to the confidence scores;
receive structured data associated with a selected reference record from the ranked list, and generate, by inputting the structured data into a second neural network comprising a convolutional neural network architecture, an analysis feature vector comprising a plurality of category labels and associated weight values; and
generate, by inputting the analysis feature vector and the set of classification labels into a data generation model, output data identifying a ranked subset of the reference records, and transmit the output data to the client terminal via the packet-switched network.

2. The system according to claim 1, wherein the circuitry is further configured to store the input feature vector in the database as a time-series record associated with the user, and to retrieve previously stored time-series records to detect a trend in the numerical measurements over a plurality of sessions.

3. The system according to claim 1, wherein the structured data comprises a plurality of modality-specific data elements including at least one of a numerical vector, a three-dimensional image tensor, or a two-dimensional image tensor, and wherein the second neural network processes each modality-specific data element using a corresponding feature extraction pipeline.

4. The system according to claim 1, wherein the circuitry is further configured to query a specialist database comprising qualification records, service history counts, evaluation scores, and geographic accessibility data, and to generate the output data by applying the data generation model to the analysis feature vector and the queried specialist database records.

5. The system according to claim 1, wherein the input feature vector encodes a plurality of measurement types including at least a temperature value, a presence or absence indicator for each of a plurality of symptom categories, and a location identifier with an associated intensity value.

6. The system according to claim 1, wherein the circuitry is further configured to generate the ranked list by computing, for each reference record, a composite score based on a specialty match with the classification labels, a geographic distance to the client terminal, and an evaluation metric stored in the database.

7. The system according to claim 1, wherein the circuitry is further configured to estimate an emotion of the user by applying an emotion identification model to sensor data received from the client terminal, and to select, based on the estimated emotion, an input configuration that determines an order and a scope of the numerical measurements requested from the client terminal.

8. The system according to claim 1, wherein the circuitry is further configured to retrieve, from the database, historical input feature vectors associated with the user, and to determine an input format for the numerical measurements based on patterns identified in the historical input feature vectors.

9. The system according to claim 1, wherein the circuitry is further configured to retrieve attribute data of the user from the database, the attribute data comprising lifestyle indicators and historical records, and to apply a filtering function to the attribute data to select a subset of measurement fields to request from the client terminal.

10. The system according to claim 1, wherein the circuitry is further configured to estimate an emotion of the user by applying an emotion identification model to at least one of facial image data, voice waveform data, or text data received from the client terminal, and to determine a priority order of the numerical measurements to request from the client terminal based on the estimated emotion.

11. The system according to claim 1, wherein the circuitry is further configured to acquire geographic location data from the client terminal, and to adjust a composition of the numerical measurements requested from the client terminal based on regional characteristic data associated with the geographic location data.

12. The system according to claim 1, wherein the circuitry is further configured to retrieve social media activity data of the user via the packet-switched network, extract keyword features from the social media activity data using a natural language processing model, and supplement the input feature vector with the extracted keyword features.

13. The system according to claim 1, wherein the circuitry is further configured to estimate an emotion of the user by applying an emotion identification model to multimodal sensor data received from the client terminal, and to select, based on the estimated emotion, a branching parameter that controls a level of detail of the set of classification labels transmitted to the client terminal.

14. The system according to claim 1, wherein the circuitry is further configured to compute an importance score for each element of the input feature vector based on a severity indicator and a frequency indicator, and to adjust a granularity of the set of classification labels based on the computed importance scores.

15. The system according to claim 1, wherein the circuitry is further configured to classify the input feature vector into a category by applying a categorization model, and to select, based on the category, a specialized neural network architecture from among a plurality of neural network architectures for generating the set of classification labels.

16. The system according to claim 1, wherein the circuitry is further configured to estimate an emotion of the user by applying an emotion identification model to sensor data received from the client terminal, and to reorder the ranked list of reference records based on the estimated emotion before transmitting the ranked list to the client terminal.

17. The system according to claim 1, wherein the circuitry is further configured to assign a processing priority to the input feature vector based on a reception timestamp, such that an input feature vector having a more recent reception timestamp is processed with a higher priority.

18. A system comprising:
circuitry configured to:
receive, from a client terminal via a packet-switched network, an input feature vector of 1×N dimensions encoding a plurality of numerical measurements, where N is a number of measurement items;
normalize the input feature vector by applying Z-score normalization, perform missing value imputation, and detect outliers in the normalized input feature vector;
generate, by inputting the normalized input feature vector as a multidimensional tensor into a first neural network comprising a Transformer-based large language model with attention layers, a ReLU activation function, batch normalization, and dropout, a set of classification labels each associated with a probability score;
retrieve, from a database, a plurality of reference records corresponding to the set of classification labels, each reference record comprising a specialty field, a geographic distance value, a service history count, and an evaluation score, and generate a ranked list of the reference records by applying a scoring function that integrates the probability scores with the specialty field, the geographic distance value, and the evaluation score;
receive structured data associated with a selected reference record, the structured data comprising at least one of a numerical vector, a three-dimensional image tensor, or a two-dimensional image tensor, preprocess the structured data by performing noise removal and feature extraction, and generate, by inputting the preprocessed structured data into a second neural network comprising a convolutional neural network architecture, an analysis feature vector comprising a plurality of category labels, severity scores, and recommended action labels; and
generate, by inputting the analysis feature vector, the set of classification labels, and specialist database records into a data generation model comprising a gradient boosting decision tree or a random forest, output data identifying a ranked subset of the reference records with recommendation scores and recommendation reasons, and transmit the output data to the client terminal via the packet-switched network.

19. The system according to claim 18, wherein the circuitry is further configured to estimate an emotion of the user by inputting at least one of a facial image tensor, a voice waveform tensor, or text data received from the client terminal into an emotion identification model comprising an attention fusion layer that integrates feature extraction results from a convolutional neural network for image data, a recurrent neural network for voice data, and a Transformer-based model for text data, and to adjust a processing flow for generating the set of classification labels based on an emotion label and a probability distribution output by the emotion identification model.

20. A method performed by circuitry of a system, the method comprising:
receiving, from a client terminal via a packet-switched network, an input feature vector encoding a plurality of numerical measurements;
generating, by inputting the input feature vector as a multidimensional tensor into a first neural network comprising a Transformer-based architecture with attention layers, a set of classification labels each associated with a confidence score;
retrieving, from a database, a plurality of reference records corresponding to the set of classification labels, and generating a ranked list of the reference records by applying a scoring function to the confidence scores;
receiving structured data associated with a selected reference record from the ranked list, and generating, by inputting the structured data into a second neural network comprising a convolutional neural network architecture, an analysis feature vector comprising a plurality of category labels and associated weight values; and
generating, by inputting the analysis feature vector and the set of classification labels into a data generation model, output data identifying a ranked subset of the reference records, and transmitting the output data to the client terminal via the packet-switched network.
